(19) United States
(12) Patent Application Publication
(10) Pub. No.: US 2008/0015196 A1
(43) Pub. Date: Jan. 17, 2008

(54) Imidazopyrazines, Imidazopyridines, and Imidazopyrimidines as Crf1 Receptor Ligands

(75) Inventors: Dario Doller, Wallingford, CT (US); Lu Yan Zhang, Branford, CT (US); Ping Ge, Durham, CT (US); Yasuchika Yamaguchi, Guilford, CT (US); Raymond F. Horvath, Guildford, CT (US); Xuechun Zhang, Branford, CT (US)

Correspondence Address: EDWARDS ANGELL PALMER & DODGE LLP, P.O. BOX 55874, BOSTON, MA, 02205, US

(73) Assignee: NEUROGEN CORPORATION, BRANFORD, CT (US)

(21) Appl. No.: 11/578,254

(22) PCT Filed: Apr. 15, 2005
(86) PCT No.: PCT/US05/13049

§ 371 Date: Oct. 12, 2006

Related U.S. Application Data
(60) Provisional application No. 60/563,239, filed on Apr. 16, 2004.
(60) Provisional application No. 60/607,211, filed on Sep. 3, 2004.

(51) Int. Cl. A61K 31/495; C07D 471/00; C07D 487/02; G01N 33/566

(52) U.S. Cl. 514/249; 436/501; 514/303; 544/280; 544/295; 544/350; 546/117

(57) ABSTRACT

Novel aryl substituted imidazopyrazines, imidazopyrimidines, and imidazopyridines are provided. Such compounds can act as selective modulators of CRF receptors. The imidazopyrazines, imidazopyrimidines, and imidazopyridines compounds provided herein are useful in the treatment of a number of CNS and periphereal disorders, particularly stress, anxiety, depression, cardiovascular disorders, and eating disorders. Methods of treatment of such disorders and well as packaged pharmaceutical compositions are also provided. Compounds provided are also useful as probes for the localization of CRF receptors and as standards in assays for CRF receptor binding. Methods of using the compounds in receptor localization studies are given.

### FIELD OF THE INVENTION

The present invention relates to compounds that modulate a CRF (Corticotropin Releasing Factor) receptor. Certain imidazopyrazine, imidazopyridine, and imidazopyrimidine compounds provided herein bind with high selectivity and/or high affinity to CRF receptors, preferably CRF1 receptors. This invention also relates to pharmaceutical compositions comprising such compounds and to the use of such compounds in treatment of psychiatric disorders and neurological diseases, including major depression, anxiety-related disorders, post-traumatic stress disorder, supranuclear palsy and feeding disorders, as well as treatment of immunological, cardiovascular or heart-related diseases and colonic hypersensitivity associated with psychopathological disturbance and stress. Additionally this invention provides labeled probes for the localization of CRF receptors in cells and tissues.

### BACKGROUND OF THE INVENTION

Corticotropin releasing factor (CRF), a 41 amino acid peptide, is the primary physiological regulator of proopiomelanocortin (POMC) derived peptide secreted from the anterior pituitary gland. In addition to its endocrine role at the pituitary gland, immunohistochemical localization of CRF has demonstrated that the hormone has a broad extrahypothalamic distribution in the central nervous system and produces a wide spectrum of autonomic, electrophysiological and behavioral effects consistent with a neurotransmitter or neuromodulator role in brain. There is also evidence that CRF plays a significant role in integrating the response of the immune system to physiological, psychological, and immunological stressors.

CRF acts by binding to and modulating the signal transduction activities of specific cell surface receptors, including CRF1 receptors and CRF2 receptors. These receptors are found at high concentrations in the central nervous system (CNS), particularly in certain regions of the brain. CRF1 receptors are also found outside the CNS.

Clinical data provide evidence that CRF has a role in psychiatric disorders and neurological diseases including depression, anxiety-related disorders and feeding disorders. A role for CRF has also been postulated in the etiology and pathophysiology of Alzheimer's disease, Parkinson's disease, Huntington's disease, progressive supranuclear palsy and amyotrophic lateral sclerosis as they relate to the dysfunction of CRF neurons in the central nervous system.

In affective disorder, or major depression, the concentration of CRF is significantly increased in the cerebral spinal fluid (CSF) of drug-free individuals. Furthermore, the density of CRF receptors is significantly decreased in the frontal cortex of suicide victims, consistent with a hypersecretion of CRF. In addition, there is a blunted adrenocorticotropin (ACTH) response to CRF (i.v. administered) observed in depressed patients. Preclinical studies in rats and non-human primates provide additional support for the hypothesis that hypersecretion of CRF may be involved in the symptoms seen in human depression. There is also preliminary evidence that tricyclic antidepressants can alter CRF levels and thus modulate the numbers of CRF receptors in brain.

The mechanisms and sites of action through which conventional anxiolytics and antidepressants produce their therapeutic effects remain to be fully elucidated. It has been hypothesized however, that they are involved in the suppression of CRF hypersecretion that is observed in these disorders.

CRF has been implicated in the etiology of anxiety-related disorders. CRF produces anxiogenic effects in animals and interactions between benzodiazepine/non-benzodiazepine anxiolytics and CRF have been demonstrated in a variety of behavioral anxiety models. Preliminary studies using the putative CRF receptor antagonist α-helical ovine CRF (9-41) in a variety of behavioral paradigms demonstrate that the antagonist produces “anxiolytic-like” effects that are qualitatively similar to the benzodiazepines. Neurochemical, endocrine and receptor binding studies have all demonstrated interactions between CRF and benzodiazepine anxiolytics providing further evidence for the involvement of CRF in these disorders. Chlordiazepoxide attenuates the “anxiogenic” effects of CRF in both the conflict test and in the acoustic startle test in rats. The benzodiazepine receptor antagonist Ro 15-1788, which was without behavioral activity alone in the operant conflict test, reversed the effects of CRF in a dose-dependent manner, while the benzodiazepine inverse agonist FG 7142 enhanced the actions of CRF.

CRF activity has also been implicated in the pathogeneisis of certain cardiovascular or heart-related, digestive, degenerative, dermatological, and immunological, diseases and disorders such as hypertension, tachycardia and congestive heart failure, stroke, acne and osteoporosis, as well as in premature birth, psychosocial dwarfism, stress-induced fever, ulcer, diarrhea, post-operative ileus and colonic hypersensitivity, e.g., associated with psychopathological disturbance and stress.

### SUMMARY OF THE INVENTION

The invention provides aryl substituted imidazopyrazines, imidazopyrimidines, and imidazopyridines, including compounds of Formula I (shown below). Such compounds bind to cell surface receptors, preferably G-coupled protein receptors, especially CRF receptors and most preferably CRF1 receptors. Preferred compounds of the invention exhibit high affinity for CRF1 receptors, i.e., they bind to, activate, inhibit, or otherwise modulate the activity of receptors other than CRF receptors with affinity constants of less than 1 micromolar, preferably less than 100 nanomolar, and most preferably less than 10 nanomolar. Additionally, preferred compounds also exhibit high selectivity for CRF1 receptors.

The invention further comprises methods of treating patients suffering from certain diseases or disorders by administering to such patients an amount of a compound disclosed herein effective to reduce signs or symptoms of the disease or disorder. These diseases and disorders include CNS disorders, particularly affective disorders, anxiety, stress, depression, eating disorders, substance abuse, and also include certain digestive disorders, particularly irritable bowel syndrome and Crohn's disease. These diseases or disorders further include cardiovascular or heart-related, digestive, degenerative, dermatological, and immunological, diseases and disorders such as hypertension, tachycardia and congestive heart failure, stroke, acne and osteoporosis, as well as premature birth, psychosocial dwarfism, stress-induced fever, ulcer, diarrhea, post-operative ileus and colonic hypersensitivity. The patient suffering from such diseases or disorders may be a human or other animal (preferably a mammal), such as a domesticated companion animal (pet) or a livestock animal. In certain aspects the invention provides for treatment with a compound disclosed herein for prophylaxis of diseases and disorders.

Preferred compounds disclosed herein for such therapeutic and prophylactic purposes are those that antagonize the binding of CRF to CRF receptors (preferably CRF1 and/or CRF2, or more preferably CRF1 receptors). The ability of compounds to act as antagonists can be measured as an IC₅₀ value as described below.

According to yet another aspect, the present invention provides pharmaceutical compositions comprising a compound disclosed herein or pharmaceutically acceptable salts or solvates thereof together with at least one pharmaceutically acceptable carrier or excipient, which compositions are useful for the treatment or prophylaxis of the disorders recited above. The invention further provides methods of treating patients suffering from any of these disorders with an effective amount of such a compound or composition.

Additionally this invention relates to the use of labeled compounds (particularly radiolabeled instances of compounds disclosed herein) as probes for the localization of CRF receptors in cells and tissues and as standards and reagents for use in determining the receptor-binding characteristics of other (test) compounds.

Thus, in a first aspect, the invention provides compounds of Formula I and the pharmaceutically acceptable salts thereof.

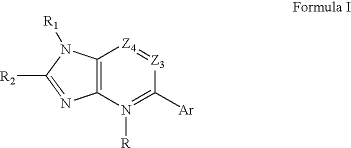

Ar, in Formula I, is chosen from: phenyl which is mono-, di-, or tri-substituted, 1-naphthyl and 2-naphthyl, each of which is optionally mono-, di-, or tri-substituted, and optionally mono-, di-, or tri-substituted heteroaryl, having from 1 to 3 rings, 5 to 7 ring members in each ring and, in at least one ring, from 1 to about 3 heteroatoms selected from N, O, and S;

R is oxygen, methyl, or absent.

R₁ is chosen from optionally substituted alkyl, optionally substituted alkenyl, optionally substituted alkynyl, optionally substituted cycloalkyl, optionally substituted (cycloalkyl)alkyl, optionally substituted mono- or di-alkylamino, optionally substituted alkanoyl, optionally substituted aryl, and optionally substituted heteroaryl, having from 1 to 3 rings, 5 to 7 ring members in each ring and, in at least one ring, from 1 to about 3 heteroatoms selected from N, O, and S.

R₂ is chosen from hydrogen, halogen, hydroxy, amino, cyano, nitro, alkyl, alkoxy, haloalkyl, haloalkoxy, aminoalkyl, and mono- and dialkylamino.

Z₃ is nitrogen or CR₃; Z₄ is nitrogen or CR₄; and Z₃ and Z₄ are not both nitrogen.

R₃ and R₄ are independently chosen from hydrogen, halogen, hydroxy, amino, cyano, nitro, optionally substituted alkyl, optionally substituted alkenyl, optionally substituted alkynyl, optionally substituted alkoxy, optionally substituted mono- and di-alkylamino, optionally substituted (cycloalkyl)alkyl, optionally substituted (cycloalkyl)oxy, optionally substituted (cycloalkyl)alkoxy, optionally substituted alkylthio, optionally substituted alkylsulfinyl, optionally substituted alkylsulfonyl, optionally substituted mono- and di-alkylcarboxamide, optionally substituted aryl, and optionally substituted heteroaryl, having from 1 to 3 rings, 5 to 7 ring members in each ring and, in at least one ring, from 1 to about 3 heteroatoms selected from the group consisting of N, O, and S.

### DETAILED DESCRIPTION OF THE INVENTION

Chemical Description and Terminology

Compounds are generally described herein using standard nomenclature.

Certain compounds described herein contain one or more asymmetric elements such as stereogenic centers, stereogenic axes and the like (e.g., asymmetric carbon atoms) so that the compounds can exist in different stereoisomeric forms. These compounds can be, for example, racemates or optically active forms. For compounds with two or more asymmetric elements, these compounds can additionally be mixtures of diastereomers. Unless otherwise specified all optical isomers and mixtures thereof are encompassed for compounds having asymmetric centers. In addition, compounds with carbon-carbon double bonds may occur in Z- and E-forms, with all isomeric forms of the compounds being included in the present invention unless otherwise specified. Where a compound exists in various tautomeric forms, the invention is not limited to any one of the specific tautomers, but rather encompasses all tautomeric forms.

The present invention is intended to include all isotopes of atoms occurring in the present compounds. Isotopes include those atoms having the same atomic number but different mass numbers. By way of general example, and without limitation, isotopes of hydrogen include tritium and deuterium and isotopes of carbon include ¹¹C, ¹³C, and ¹⁴C. Preferred compounds for therapeutic uses are not radiolabelled. That is, these compounds comprise naturally occurring mixtures of isotopes present in nature for each element, e.g., carbon is typically comprises about 99% ¹²C, 1% ¹³C, and trace amounts of ¹⁴C. Preferred compounds of the invention, particularly compounds suitable for use in the imaging methods, include one or more radioisotopes capable of emitting one or more forms of radiation which are suitable for detection with any standard radiology equipment such as PET, SPECT, gamma cameras, MRI and the like. Preferred radioisotopes include tritium and isotopes of carbon, fluorine, technetium, iodine and other isotopes capable of emitting positrons. Particularly preferred radioisotopes include ³H, ¹¹C, ¹⁸F, ³²P, ⁹⁹Tc, and ¹²³I

Certain compounds are described herein using a general formula, such as Formula I, which includes variables, such as Ar, R¹, and R². Unless otherwise specified, each variable within such a formula is defined independently of other variables. Thus, for example, if a group is shown to be substituted with 0-2 R*, then said group may optionally be substituted with up to two R* groups and R* at each occurrence is selected independently from the definition of R*. Also, combinations of substituents and/or variables are permissible only if such combinations result in stable compounds.

A “substituent,” as used herein, refers to a molecular moiety that is covalently bonded to an atom within a molecule of interest. For example, a “ring substituent” may be a moiety such as a halogen, alkyl group, haloalkyl group or other substituent discussed herein that is covalently bonded to an atom (preferably a carbon or nitrogen atom) that is a ring member. The term “substituted,” as used herein, means that any one or more hydrogens on the designated atom is replaced with a selection from the indicated substituents, provided that the designated atom's normal valence is not exceeded, and that the substitution results in a stable compound (i.e., a compound that can be isolated, characterized and tested for biological activity). When a substituent is oxo (i.e., ═O), then 2 hydrogens on the atom are replaced. When aromatic moieties are substituted by an oxo group, the aromatic ring is replaced by the corresponding partially unsaturated ring. For example a pyridyl group substituted by oxo is a tetrahydropyridone.

The phrase “optionally substituted” indicates that a group may either be unsubstituted or substituted at one or more of any of the available positions, typically 1, 2, 3, 4, or 5 positions, by one or more suitable substituents such as those disclosed herein. Various groups within the compounds and formulae set forth herein are “optionally substituted” including, for example, R¹, R², and Ar. Optional substitution may also be indicated by the phrase “substituted with from 0 to X substituents,” in which X is the maximum number of substituents.

Suitable substituents include, for example, halogen, cyano, amino, hydroxy, nitro, azido, carboxamido, —COOH, SO₂NH₂, alkyl (e.g., C₁-C₈alkyl), alkenyl (e.g., C₂-C₈alkenyl), alkynyl (e.g., C₂-C₈alkynyl), alkoxy (e.g., C₁-C₈alkoxy), alkyl ether (e.g., C₂-C₈alkyl ether), alkylthio (e.g., C₁-C₈alkylthio), mono- or di-(C₁-C₈alkyl)amino, haloalkyl (e.g., C₁-C₆haloalkyl), hydroxyalkyl (e.g., C₁-C₆hydroxyalkyl), aminoalkyl (e.g., C₁-C₆aminoalkyl), haloalkoxy (e.g., C₁-C₆haloalkoxy), alkanoyl (e.g., C₁-C₈alkanoyl), alkanone (e.g., C₁-C₈alkanone), alkanoyloxy (e.g., C₁-C₈alkanoyloxy), alkoxycarbonyl (e.g., C₁-C₈alkoxycarbonyl), mono- and di-(C₁-C₈alkyl)amino, mono- and di-(C₁-C₈alkyl)aminoC₁-C₈alkyl, mono- and di-(C₁-C₈alkyl)carboxamido, mono- and di-(C₁-C₈alkyl)sulfonamido, alkylsulfinyl (e.g., C₁-C₈alkylsulfinyl), alkylsulfonyl (e.g., C₁-C₈alkylsulfonyl), aryl (e.g., phenyl), arylalkyl (e.g., (C₆-C₁₈aryl)C₁-C₈alkyl, such as benzyl and phenethyl), aryloxy (e.g., C₆-C₁₈aryloxy such as phenoxy), arylalkoxy (e.g., (C₆-C₁₈aryl)C₁-C₈alkoxy) and/or 3- to 8-membered heterocyclic groups. Certain groups within the formulas provided herein are optionally substituted with from 1 to 3, 1 to 4 or 1 to 5 independently selected substituents.

A dash (“-”) that is not between two letters or symbols is used to indicate a point of attachment for a substituent. For example, —CONH₂ is attached through the carbon atom.

As used herein, “alkyl” is intended to include both branched and straight-chain saturated aliphatic hydrocarbon groups, and where specified, having the specified number of carbon atoms. Thus, the term C₁-C₆alkyl, as used herein, indicates an alkyl group having from 1 to 6 carbon atoms. “C₀-C₄alkyl” refers to a bond or a C₁-C₄alkyl group. Alkyl groups include groups having from 1 to 8 carbon atoms (C₁-C₈alkyl), from 1 to 6 carbon atoms (C₁-C₆alkyl) and from 1 to 4 carbon atoms (C₁-C₄alkyl), such as methyl, ethyl, n-propyl, isopropyl, n-butyl, sec-butyl, tert-butyl, pentyl, 2-pentyl, isopentyl, neopentyl, hexyl, 2-hexyl, 3-hexyl, and 3-methylpentyl. “Aminoalkyl” is an alkyl group as defined herein substituted with one or more —NH₂ groups.

“Carbhydryl” is a hydrocarbon group that may be straight branched or cyclic and contain 0 or 1 or more double or triple bonds. Preferred carbhydryl groups are C₁-C₆ or more preferably C₁-C₄ carbhydryl groups. Preferred carbhydryl groups are straight or branched groups. “Hydroxyalkyl” is a hydroxy group as defined herein substituted with one or more —H groups.

“Alkenyl” refers to a straight or branched hydrocarbon chain comprising one or more unsaturated carbon-carbon bonds, such as ethenyl and propenyl. Alkenyl groups include C₂-C₈alkenyl, C₂-C₆alkenyl and C₂-C₄alkenyl groups (which have from 2 to 8, 2 to 6 or 2 to 4 carbon atoms, respectively), such as ethenyl, allyl or isopropenyl.

“Alkynyl” refers to straight or branched hydrocarbon chains comprising one or more triple carbon-carbon bonds. Alkynyl groups include C₂-C₈alkynyl, C₂-C₆alkynyl and C₂-C₄alkynyl groups, which have from 2 to 8, 2 to 6 or 2 to 4 carbon atoms, respectively. Alkynyl groups include for example groups such as ethynyl and propynyl.

“Alkoxy” represents an alkyl group as defined above with the indicated number of carbon atoms attached through an oxygen bridge. Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy.

The term “alkanoyl” refers to an acyl group in a linear or branched arrangement (e.g., —(C═O)-alkyl). Alkanoyl groups include C₂-C₈alkanoyl, C₂-C₆alkanoyl and C₂-C₄alkanoyl groups, which have from 2 to 8, 2 to 6, or 2 to 4 carbon atoms, respectively. “C₁alkanoyl” refers to —C═O)—H, which (along with C₂-C₈alkanoyl) is encompassed by the term “C₁-C₈alkanoyl.”

As used herein, the term “alkylthio” refers to an alkyl group attached via a thioether linkage. Alkylthio groups include C₁-C₈alkylthio, C₁-C₆alkylthio and C₁-C₄alkylthio, which have from 1 to 8, 1 to 6 or 1 to 4 carbon atoms, respectively.

“Alkylsulfinyl,” as used herein, refers to an alkyl group attached via a sulfinyl linkage. Alkylsulfinyl groups include C₁-C₈alkylsulfinyl, C₁-C₆alkylsulfinyl, and C₁-C₄alkylsulfinyl, which have from 1 to 8, 1 to 6, and 1 to 4 carbon atoms, respectively.

By “alkylsulfonyl,” as used herein, is meant an alkyl group attached via a sulfonyl linkage. Alkylsulfonyl groups include C₁-C₈alkylsulfonyl, C₁-C₆alkylsulfonyl, and C₁-C₄alkylsulfonyl, which have from 1 to 8, 1 to 6, and 1 to 4 carbon atoms, respectively.

“Alkylamino” refers to a secondary or tertiary amine having the general structure —NH-alkyl or —N(alkyl)(alkyl), wherein each alkyl may be the same or different. Such groups include, for example, mono- and di-(C₁-C₈alkyl)amino groups, in which each alkyl may be the same or different and may contain from 1 to 8 carbon atoms, as well as mono- and di-(C₁-C₆alkyl)amino groups and mono- and di-(C₁-C₄alkyl)amino groups. Alkylaminoalkyl refers to an alkylamino group linked via an alkyl group (i.e., a group having the general structure -alkyl-NH-alkyl or -alkyl-N(alkyl)(alkyl)). Such groups include, for example, mono- and di-(C₁-C₈alkyl)aminoC₁-C₈alkyl, mono- and di-(C₁-C₆alkyl)aminoC₁-C₆alkyl, and mono- and di-(C₁-C₄alkyl)aminoC₁-C₄alkyl, in which each alkyl may be the same or different.

The term “carboxamide” or “amido” refers to an amide group (i.e., —(C═O)NH₂). “Alkylcarboxamide” refers to —NHC(═O)alkyl, preferably —NHC(═O)C₁-C₂alkyl.

The term “cycloalkyl” refers to hydrocarbon ring groups, having the specified number of carbon atoms, usually from 3 to about 8 ring carbon atoms, or from 3 to about 7 ring carbon atoms. Cycloalkyl groups include C₃-C₈, and C₃-C₇ cycloalkyl groups, which have from 3 to 8 and 3 to 7 carbon atoms, respectively. Examples of cycloalkyl groups include cyclopropyl, cyclobutyl, cyclopentyl, and cyclohexyl groups, as well as bridged and caged saturated ring groups such as norbornane or adamantane and the like.

In the term “(cycloalkyl)alkyl,” “cycloalkyl” and “alkyl” are as defined above, and the point of attachment is on the alkyl group. This term encompasses, but is not limited to, cyclopropylmethyl, cyclohexylmethyl, and cyclohexylethyl.

The term “halogen” or “halo” indicates fluorine, chlorine, bromine, or iodine.

“Haloalkyl” refers to both branched and straight-chain saturated aliphatic hydrocarbon groups having the specified number of carbon atoms, substituted with 1 or more halogen atoms. Examples of haloalkyl include, but are not limited to, trifluoromethyl, difluoromethyl, 2-fluoroethyl, and penta-fluoroethyl.

“Haloalkoxy” indicates a haloalkyl group as defined above attached through an oxygen bridge.

As used herein, the term “aryl” indicates aromatic groups containing only carbon in the aromatic ring(s). Such aromatic groups may be further substituted with carbon or non-carbon atoms or groups. Typical aryl groups contain 1 to 3 separate or fused rings, at least one of which is aromatic, and from 6 to about 18 ring atoms, without heteroatoms as ring members. Specifically preferred carbocyclic aryl groups include phenyl and napthyl, including 1-naphthyl and 2-naphthyl. When indicated, carbon atoms present within a carbocyclic ring may be optionally substituted with any of variety of ring substituents, as described above, or with specifically listed substituents.

The term “arylalkyl” refers to an aryl group is linked via an alkyl group. Certain arylalkyl groups are (C₆-C₁₈aryl)C₁-C₈alkyl groups (i.e., groups in which a 6- to 18-membered aryl group is linked via a C₁-C₈alkyl group). Such groups include, for example, groups in which phenyl or naphthyl is linked via a bond or C₁-C₈alkyl, preferably via C₁-C₄alkyl, such as benzyl, 1-phenyl-ethyl, 1-phenyl-propyl and 2-phenyl-ethyl.

The term “aryloxy” refers to an aryl group linked via a carbonyl (i.e., a group having the general structure —C(═O)—O-aryl). Phenoxy is a representative aryloxy group.

As used herein, the term “heteroaryl” is intended to indicate a stable 5- to 7-membered monocyclic or bicyclic or 7- to 10-membered bicyclic heterocyclic ring which contains at least 1 aromatic ring that contains from 1 to 4 heteroatoms selected from N, O, and S, with remaining ring atoms being carbon. When the total number of S and O atoms in the heteroaryl group exceeds 1, then these heteroatoms are not adjacent to one another. It is preferred that the total number of S and O atoms in the heterocycle is not more than 1, 2, or 3, more typically 1 or 2. It is particularly preferred that the total number of S and 0 atoms in the aromatic heterocycle is not more than 1. Examples of heteroaryl groups include pyridyl, furanyl, indolyl, pyrimidinyl, pyridizinyl, pyrazinyl, imidazolyl, oxazolyl, thienyl, thiazolyl, triazolyl, isoxazolyl, quinolinyl, pyrrolyl, pyrazolyl, and 5,6,7,8-tetrahydroisoquinoline.

The term “heterocyclic group” or “heterocycle” is used to indicate saturated, partially unsaturated, or aromatic groups having 1 or 2 rings, 3 to 8 atoms in each ring and in at least one ring between 1 and 3 heteroatoms selected from N, O, and S. Any nitrogen or sulfur heteroatoms may optionally be oxidized. The heterocyclic group may be attached to its pendant group at any heteroatom or carbon atom that results in a stable structure. The heterocyclic groups described herein may be substituted on a carbon or nitrogen atom if the resulting compound is stable. A nitrogen atom in the heterocycle may optionally be quaternized.

Representative examples of heteroaryl groups and heterocyclic groups include, but are not limited to, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, NH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl;-1,2,5oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

“A CRF receptor” is a G-coupled protein receptor that specifically binds CRF peptide. Preferably the CRF receptor is a mammalian CRF1 receptor.

A “CRF receptor modulator” is a compound that affects signal transduction by CRF receptors. Preferably the CRF receptor modulator affects signal transduction by binding to CRF receptors and either increasing or inhibiting signal transduction by CRF receptors.

A “CRF antagonist” is a compound that binds to CRF receptors, inhibiting CRF binding to the CRF receptor, and decreasing CRF mediated CRF receptor signal transduction. CRF receptor binding can be quantitated using a standard CRF radioligand binding assay such as the assay provided in Example 8.

A “therapeutically effective amount” of a compound is an amount that is sufficient to result in a discernible patient benefit. For example, a therapeutically effective amount may reduce symptom severity or frequency. Alternatively, or in addition, a therapeutically effective amount may improve patient outcome and/or prevent or delay disease or symptom onset.

As used herein, a “pharmaceutically acceptable salt” is an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)ₙ—COOH where n is 0-4 and the like. Similarly, pharmaceutically acceptable cations include, but are not limited to sodium, potassium, calcium, aluminum, lithium and ammonium. Those of ordinary skill in the art will recognize further pharmaceutically acceptable salts for the compounds provided herein, including those listed by Remington's Pharmaceutical Sciences, 17th ed., Mack Publishing Company, Easton, Pa., p. 1418 (1985). Accordingly, the present disclosure should be construed to include all pharmaceutically acceptable salts of the compounds set forth herein. Additionally, the term “pharmaceutically acceptable salt” is meant to encompass pharmaceutically acceptable prodrugs, hydrates or clathrates of such compounds.

A wide variety of synthetic procedures is available for the preparation of pharmaceutically acceptable acid or base salts. In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in water, an organic solvent, or a mixture of the two; generally, nonaqueous media like ether, ethyl acetate, ethanol, isopropanol or acetonitrile are preferred.

A “prodrug” is a compound that may not fully satisfy the structural requirements of the compounds provided herein, but is modified in vivo, following administration to a patient, to produce such an aryl substituted imidazopyrazine, imidazopyrimidine, or imidazopyridine. For example, a prodrug may be an acylated derivative of a compound as provided herein. Prodrugs include compounds wherein hydroxy, amine, or sulfhydryl groups are bonded to any group that, when administered to a mammalian subject, cleaves to form a free hydroxyl, amino, or sulfhydryl group, respectively. Examples of prodrugs include, but are not limited to, acetate, formate, and benzoate derivatives of alcohol and amine functional groups within the compounds provided herein. Preferred prodrugs include acylated derivatives. Prodrugs may be prepared by modifying functional groups present in the compounds in such a way that the modifications are cleaved to the parent compounds. Those of ordinary skill in the art will recognize various synthetic methods that may be employed to prepare prodrugs of the compounds provided herein.

A “patient” is any individual treated with a CRF receptor modulator as provided herein. Patients include humans, as well as other animals such as companion animals (e.g., dogs and cats) and livestock. Patients may be experiencing one or more symptoms of a condition responsive to CRF receptor modulation, or may be free of such symptom(s) (i.e., treatment may be prophylactic).

CRF Receptor Modulators

As noted above, the present invention provides CRF receptor modulators (i.e., compounds that modulate CRF receptor-mediated signal transduction; preferably compounds that also detectably bind to CRF receptors), preferably CRF1 receptor modulators. CRF receptor modulators may be used to modulate CRF receptor activity in a variety of contexts, including in the treatment of patients suffering from diseases or disorders responsive to CRF receptor modulation, such as stress disorders and digestive disorders. CRF receptor modulators disclosed herein may also be used within a variety of in vitro assays (e.g., assays for receptor activity), as probes for detection and localization of CRF receptors, particularly CRF1 receptors, and as standards in assays of ligand binding and CRF receptor-mediated signal transduction.

CRF receptor modulators provided herein are aryl substituted imidazopyrazines, imidazopyrimidines, or imidazopyridines (as well as pharmaceutically acceptable salts thereof) that detectably alter, preferably decrease, CRF receptor activation and/or signal transduction activity at submicromolar concentrations. Such an alteration in CRF receptor activity may be measured using a standard in vitro CRF receptor-radioligand binding assay (Example 8). The present invention is based, in part, on the discovery that small molecules of Formula I and Formula V act as antagonists of CRF1 receptors.

In addition to the compounds of Formula I described above, the invention further provides compounds of Formula I, and pharmaceutically acceptable salts thereof, in which the variables, R, R₁, R₂, Z₃, Z₄, and Ar are as defined below. Such compounds, which have the same general chemical formula as compounds of Formula I, but differ in the definitions assigned the variables, will be referred to as compounds of Formula IA.

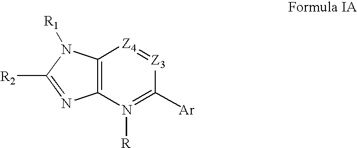

R is oxygen, methyl, or absent. Preferably R is absent.

Ar is chosen from: phenyl which is mono-, di-, or tri-substituted with R_{A}, and 1-naphthyl, 2-naphthyl, pyridyl, pyrimidinyl, pyrazinyl, pyridizinyl, thienyl, thiazolyl, oxazolyl, isoxazolyl, pyrrolyl, furanyl, and triazolyl, each of which is optionally mono-, di-, or tri-substituted with R_{A}.

R₁ is chosen from

i) C₁-C₁₀-carbhydryl, (C₃-C₇cycloalkyl)C₀-C₄carbhydryl, (C₁C₆)haloalkyl, and mono- and di-(C₁C₆)alkylamino, C₂-C₆alkanoyl; each of which is substituted with 0 or more substituents independently chosen from halogen, hydroxy, amino, oxo, cyano, C₁-C₄alkoxy, and mono- and di(C₁-C₄)alkylamino, and

ii) phenyl which is mono-, di-, or tri-substituted with R_{A}, 1-naphthyl, 2-naphthyl, pyridyl, dihydropyridyl, tetrahydropyridyl, pyrimidinyl, pyrazinyl, pyridizinyl, thienyl, thiazolyl, oxazolyl, isoxazolyl, pyrrolyl, furanyl, and triazolyl, each of which is optionally mono-, di-, or tri-substituted with R_{A},

R₂ is chosen from hydrogen, halogen, hydroxy, amino, cyano, nitro, C₁-C₆alkyl, (C₁-C₆)haloalkyl, (C₁-C₆)haloalkoxy, C₁-C₆alkoxy, amino(C₁-C₆)alkyl, and mono- and di-(C₁-C₆)alkylamino.

Z₃ is nitrogen or CR₃; Z₄ is nitrogen or CR₄; and Z₃ and Z₄ are not both nitrogen.

R₃ and R₄ independently chosen from

i) hydrogen, halogen, hydroxy, cyano, nitro, amino,

ii) C₁-C₆alkyl, C₂-C₆alkenyl, C₂-C₆alkynyl, C₁-C₆alkoxy, mono- and di-C₁-C₆alkylamino, (C₃-C₇cycloalkyl)C₀-C₄alkyl, (C₃-C₇cycloalkyl)C₀-C₄alkoxy, C₁-C₆haloalkyl, C₁-C₆haloalkoxy, —S(O)ₙ(C₁-C₆alkyl), and mono- and di-C₁-C₆alkylcarboxamide, each of which is substituted with 0 or more substituents independently chosen from halogen, amino, hydroxy, oxo, cyano, C₁-C₄alkoxy, and mono- and di-(C₁-C₄)alkylamino; and

iii) phenyl, pyridyl, pyrimidinyl, pyrazinyl, pyridizinyl, thienyl, thiazolyl, oxazolyl, isoxazolyl, pyrrolyl, furanyl, and triazolyl, each of which is substituted with 0 or more substituents independently chosen from halogen, amino, hydroxy, oxo, cyano, C₁-C₄alkoxy, and mono- and di-(C₁-C₄)alkylamino.

R_{A} is independently selected at each occurrence from halogen, cyano, nitro, hydroxy, amino, C₁-C₆haloalkyl, C₁-C₆haloalkoxy, C₁-C₆alkyl substituted with 0-2 R_{B}, C₂-C₆alkenyl substituted with 0-2 R_{B}, C₂-C₆alkynyl substituted with 0-2 R_{B}, C₃-C₇cycloalkyl substituted with 0-2 R_{B}, (C₃-C₇cycloalkyl)C₁-C₄alkyl substituted with 0-2 R_{B}, C₁-C₆alkoxy substituted with 0-2 R_{B}, —NH(C₁-C₆alkyl) substituted with 0-2 R_{B}, —N(C₁-C₆alkyl)(C₁-C₆alkyl) of which each C₁-C₆alkyl is independently substituted with 0-2 R_{B}, —XR_{C}, and Y;

R_{B} is independently selected at each occurrence from halogen, hydroxy, cyano, amino, C₁-C₄alkyl, mono- and di-(C₁-C₄)alkylamino, —S(O)ₙ(alkyl), C₁-C₄haloalkyl, C₁-C₄haloalkoxy, C₁-C₄alkanoyl, mono- and di-C₁-C₄alkylcarboxamide, —XR_{C}, and Y;

R_{C} and R_{D}, are independently selected at each occurrence from:

i) hydrogen, and

ii) C₁-C₈alkyl and C₃-C₇cycloalkyl(C₀-C₄alkyl), each of which is substituted with 0 or more substituent(s) independently selected from: oxo, hydroxy, halogen, cyano, amino, C₁-C₆alkoxy, mono- and di-C₁-C₆alkylamino, mono- and di-C₁-C₆alkylcarboxamide, —NHS(O)ₙ(C₁-C₆alkyl), —S(O)ₙ(C₁-C₆alkyl), —S(O)ₙNH(C₁-C₆alkyl), —S(O)ₙN(C₁-C₆alkyl)(C₁-C₆alkyl), and Z;

X is independently selected at each occurrence from —CH₂—, —CHR_{D}—, —O—, —C(═O)—, —C(═O)O—, —S(O)ₙ—, —NH—, —NR_{D}—, —C(═O)NH—, —C(═O)NR_{D}—, —S(O)ₙNH—, —S(O)ₙNR_{D}—, —OC(═S)S—, —NHC(═O)—, —NR_{D}C(═O)—, —NHS(O)ₙ—, and —NR_{D}S(O)ₙ—;

Y and Z are independently selected at each occurrence from: 3- to 7-membered carbocyclic or heterocyclic groups, each of which is substituted with 0 or more substituents independently selected from halogen, oxo, hydroxy, amino, cyano, C₁-C₄alkyl, C₁-C₄alkoxy, mono- and di-C₁-C₆alkylamino, and —S(O)ₙ(alkyl); and n is independently selected at each occurrence from 0, 1, and 2.

In addition to compounds of Formula I and Formula IA, the invention provides compounds of Formula II-Formula IV:

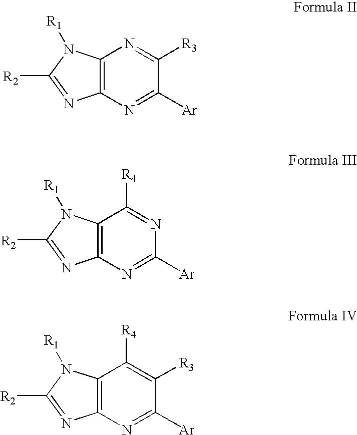

In certain embodiments, the invention provides compounds and pharmaceutically acceptable salts of Formula II through Formula IV in which the variables R₁, R₂, R₃, R₄, and Ar are defined as follows:

R₁ is chosen from C₁-C₁₀-carbhydryl, (C₃-C₇cycloalkyl)C₀-C₄-carbhydryl, and (C₁-C₆)haloalkyl, each of which is substituted with 0 or more substituents independently chosen from halogen, hydroxy, amino, oxo, cyano, C₁-C₄alkoxy, and mono- and di-(C₁-C₄)alkylamino;

R₂ is chosen from hydrogen, halogen, C₁-C₆alkyl, C₁-C₆alkoxy, C₁-C₄haloalkyl and C₁-C₄haloalkoxy, and mono- and di-C₁-C₄alkylamino;

R₃ and R₄, when present, are independently chosen from hydrogen, halogen, C₁-C₆alkyl, C₁-C₆alkoxy, C₁-C₄haloalkyl, C₁-C₄haloalkoxy, and mono- and di-C₁-C₆alkylamino;

Ar carries the definition set forth for compounds of Formula I or Formula IA.

The invention also provides compounds and pharmaceutically acceptable salts of Formula II-Formula IV in which

R₁ is chosen from C₁-C₁₀carbhydryl and (C₃-C₇cycloalkyl)C₀-C₄alkyl; each of which is substituted with one or more substituents independently chosen from halogen, hydroxy, amino, oxo, cyano, C₁-C₄alkoxy, and mono- and di-(C₁-C₄)alkylamino,

R₂ is chosen from hydrogen, halogen, C₁-C₆alkyl and C₁-C₄haloalkyl; and

R₃ and R₄, when present, are chosen from hydrogen, halogen, C₁-C₆alkyl, C₁-C₆alkoxy, (C₁-C₄)haloalkyl, (C₁-C₄)haloalkoxy, and mono- and di-C₁-C₄alkylamino.

Ar, in these embodiments, carries the definition set forth for compounds of Formula I or Formula IA.

The invention also provides compounds and salts of Formula II-Formula IV in which:

Ar is phenyl, pyridyl, or pyrimidinyl, each of which is mono-, di-, or tri-substituted with R_{A} (as defined for compounds of Formula IA);

R₁ is chosen from C₁-C₁₀carbhydryl and (C₃-C₇cycloalkyl)C₀-C₄alkyl; each of which is substituted with 0 or more substituents independently chosen from halogen, hydroxy, amino, oxo, cyano, C₁-C₄alkoxy, and mono- and di-(C₁-C₄)alkylamino,

R₂ is chosen from hydrogen, halogen, C₁-C₆alkyl and (C₁-C₄)haloalkyl; and

R₃ and R₄, when present, are chosen from hydrogen, halogen, C₁-C₆alkyl, C₁-C₆alkoxy, (C₁-C₄)haloalkyl, (C₁-C₄)haloalkoxy, and mono- and di-C₁-C₄alkylamino.

The invention further provides compounds and salts of Formula II to Formula IV in which

Ar is chosen from phenyl, pyridyl, and pyrimidinyl, each of which is mono-, di-, or tri-substituted by substituents independently chosen from:

i) halogen, cyano, (C₁-C₄)haloalkyl, (C₁-C₄)haloalkoxy, hydroxy, amino, C₃-C₇cycloalkyl(C₀-C₄alkyl), mono- and di-C₁-C₆alkylamino, —CHO, and —C(═O)CH₃,

ii) C₁-C₆alkoxy and C₁-C₆alkyl which are substituted with 0 or 2 groups independently selected from halogen, hydroxy, cyano, amino, C₁-C₄alkyl, C₁-C₄alkoxy, mono- and di-C₁-C₄alkylamino, (C₁-C₄)haloalkyl, (C₁-C₄)haloalkoxy, and C₂-C₅alkanoyl, and

iii) 3- to 7-membered carbocyclic and heterocyclic groups, each of which is substituted with 0 or more substituents independently selected from halogen, oxo, hydroxy, amino, C₁-C₄alkyl, C₁-C₄alkoxy, and mono- and di-(C₁-C₄)alkylamino; wherein at least one position ortho to the point of attachment of Ar in Formula I is substituted;

R₁ is chosen from C₁-C₁₀carbhydryl and (C₃-C₇cycloalkyl)C₀-C₄alkyl; each of which is substituted with one or more substituents independently chosen from halogen, hydroxy, amino, oxo, cyano, C₁-C₄alkoxy, and mono- and di-(C₁-C₄)alkylamino,

R₂ is chosen from hydrogen, halogen, C₁-C₆alkyl and (C₁-C₄)haloalkyl; and

R₃ and R₄, when present, are chosen hydrogen, halogen, C₁-C₆alkyl, C₁-C₆alkoxy, (C₁-C₄)haloalkyl, (C₁-C₄)haloalkoxy, mono- and di-C₁-C₄alkylamino.

In yet another embodiment the invention provides compounds and salts of Formula II-Formula IV in which

Ar is phenyl, pyridyl, or pyrimidinyl, each of which is mono-, di-, or tri-substituted with substituents independently chosen from:

i) halogen, cyano, (C₁-C₄)haloalkyl, (C₁-C₄)haloalkoxy, hydroxy, amino, C₃-C₇cycloalkylC₀-C₄alkyl, mono- and di-(C₁-C₆)alkylamino, —CHO, and —C(═O)CH₃,

ii) C₁-C₆alkoxy and C₁-C₆alkyl which are substituted with from 0 to 2 groups independently selected from halogen, hydroxy, cyano, amino, C₁-C₄alkyl, C₁-C₄alkoxy, mono- and di-(C₁-C₄)alkylamino, (C₁-C₄)haloalkyl, (C₁-C₄)haloalkoxy, and C₂-C₅alkanoyl, and

iii) phenyl, pyridyl, pyrimidinyl, pyrazinyl, morpholinyl, piperidinyl, piperazinyl, and dioxolanyl, each of which is substituted with 0 or more substituents independently selected from halogen, oxo, hydroxy, amino, C₁-C₄alkyl, C₁-C₄alkoxy, and mono- and di-(C₁-C₄)alkylamino; wherein at least one position ortho to the point of attachment of Ar in Formula I is substituted;

R₁ is chosen from C₁-C₁₀alkyl and (C₃-C₇cycloalkyl)C₀-C₄alkyl, each of which is substituted with 0 or more substituents independently chosen from halogen, hydroxy, amino, oxo, cyano, C₁-C₄alkoxy, and mono- and di-(C₁-C₄)alkylamino;

R₂ is chosen from hydrogen, halogen, C₁-C₆alkyl and (C₁-C₄)haloalkyl; and

R₃ and R₄, when present, are chosen hydrogen, halogen, C₁-C₆alkyl, C₁-C₆alkoxy, (C₁-C₄)haloalkyl, and (C₁-C₄)haloalkoxy.

Still other embodiments of the invention provide compounds and salts of Formula II-Formula IV wherein:

Ar is phenyl, pyridyl or pyrimidinyl, each of which is mono-, di-, or tri-substituted with substituents independently chosen from halogen, cyano, hydroxy, amino, C₁-C₆alkyl, C₁-C₆alkoxy, (C₁-C₂)haloalkyl, (C₁-C₂)haloalkoxy, C₃-C₇cycloalkylC₀-C₂alkyl, mono- and di-(C₁-C₄)alkylamino, —CHO, and —C(═O)CH₃;

R₁ is chosen from C₁-C₈alkyl and (C₃-C₇cycloalkyl)C₀-C₂alkyl; each of which is substituted with from 0 to 3 substituents independently chosen from halogen, hydroxy, amino, oxo, cyano, C₁-C₄alkoxy, and mono- and di-(C₁-C₄)alkylamino,

R₂ is chosen from hydrogen, halogen, C₁-C₂alkyl and (C₁-C₂)haloalkyl; and

R₃ and R₄, when present, are chosen hydrogen, halogen, C₁-C₆alkyl, C₁-C₆alkoxy, (C₁-C₂)haloalkyl, and (C₁-C₂)haloalkoxy.

Certain other embodiments of the invention provide compounds and salts of Formula II-Formula IV in which:

Ar is phenyl, pyridyl or pyrimidinyl, each of which is mono-, di-, or tri-substituted with substituents independently chosen from halogen, cyano, hydroxy, amino, C₁-C₄alkyl, C₁-C₃alkoxy, (C₁-C₂)haloalkyl, (C₁-C₂)haloalkoxy, and mono- and di-(C₁-C₄)alkylamino.

R₁ is chosen from C₁-C₈alkyl which is substituted with from 0 to 3 substituents independently chosen from halogen, hydroxy, amino, oxo, cyano, C₁-C₄alkoxy, and mono- and di-(C₁-C₄)alkylamino;

R₂ is chosen from hydrogen, halogen, C₁-C₂alkyl and (C₁-C₂)haloalkyl; and

R₃ and R₄, when present, are chosen hydrogen, halogen, C₁-C₄alkyl, C₁-C₄alkoxy, (C₁-C₂)haloalkyl, and (C₁-C₂)haloalkoxy.

The invention also provides compounds and salts of Formula II-Formula IV wherein:

Ar is phenyl, pyridyl or pyrimidinyl, each of which is mono-, di-, or tri-substituted with substituents independently chosen from halogen, amino, C₁-C₄alkyl, C₁-C₃alkoxy, (C₁-C₂)haloalkyl, (C₁-C₂)haloalkoxy, and mono- and di-(C₁-C₂)alkylamino.

R₁ is chosen from C₁-C₈alkyl which is substituted with from 0 to 3 substituents independently chosen from halogen, hydroxy, amino, C₁-C₄alkoxy, and mono- and di-(C₁-C₄)alkylamino;

R₂ is chosen from hydrogen, methyl, and ethyl; and

R₃ and R₄, when present, are chosen hydrogen, halogen, ethyl, methyl, propyl, methoxy, ethoxy, and trifluoromethyl.

Other embodiments of the invention provide compounds and pharmaceutically acceptable salts of Formula V

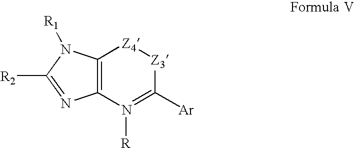

Ar, and R₁, R₂, shown in Formula V, carry the definitions set forth for these variables in Formula I.

Z₃′ is CR₃R₃′, NR₃″ or C═O; Z₄′ is CR₄R₄′, NR₄″ or C═O; and one of Z₃′ or Z₄′ is C═O;

R₃, R₃′, R₄, and R₄′ are independently chosen from hydrogen, halogen, hydroxy, amino, cyano, nitro, optionally substituted alkyl, optionally substituted alkenyl, optionally substituted alkynyl, optionally substituted alkoxy, optionally substituted mono- and di-alkylamino, optionally substituted (cycloalkyl)alkyl, optionally substituted (cycloalkyl)oxy, optionally substituted (cycloalkyl)alkoxy, optionally substituted alkylthio, optionally substituted alkylsulfinyl, optionally substituted alkylsulfonyl, optionally substituted mono- and di-alkylcarboxamide, optionally substituted aryl, and optionally substituted heteroaryl, having from 1 to 3 rings, 5 to 7 ring members in each ring and, in at least one ring, from 1 to about 3 heteroatoms selected from the group consisting of N, O, and S.

R₃″ and R₄″ are independently chosen from hydrogen, alkyl, aminoalkyl, and haloalkyl.

In addition to compounds of Formula V, described above, the invention further provides compounds of Formula V-A, and the pharmaceutically acceptable salts thereof, in which the variables, R, R₁, R₂, Z₃′, Z₄′, and Ar (as well as X, Y, Z, n, R_{A}, R_{B}, R_{C}, and R_{D}) carry the definitions set forth for these variables for compounds and salts of Formula IA. Such compounds, which have the same general chemical formula as compounds of Formula V, but differ in the definitions assigned the variables, will be referred to as compounds of Formula V-A.

R₃″ and R₄″, for compounds of Formula V-A are independently chosen from hydrogen, C₁-C₆alkyl, amino(C₁-C₆)alkyl, and (C₁-C₆)haloalkyl.

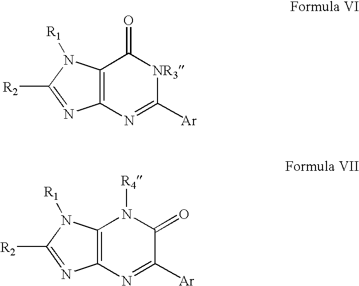

The invention further pertains to compounds and pharmaceutically acceptable salts of Formula VI and Formula VII in which Ar carries the definition set forth for compounds of Formula I or more preferably for compounds of Formula IA.

R₁ in Formula VI and Formula VII, is chosen from C₁-C₁₀carbhydryl, (C₃-C₇cycloalkyl)C₀-C₄carbhydryl, and (C₁C₆)haloalkyl, each of which is substituted with 0 or more substituents independently chosen from halogen, hydroxy, amino, oxo, cyano, C₁-C₄alkoxy, and mono- and di-(C₁-C₄)alkylamino, and

R₂ is chosen from hydrogen, halogen, C₁-C₆alkyl, C₁-C₆alkoxy, C₁-C₄haloalkyl and C₁-C₄haloalkoxy, and mono- and di-C₁-C₄alkylamino;

R₃, R₃′, R₄, and R₄′, when present, are independently chosen from hydrogen, halogen, C₁-C₆alkyl, C₁-C₆alkoxy, C₁-C₄haloalkyl, C₁-C₄haloalkoxy, and mono- and di-C₁-C₆alkylamino; and

R₃″ and R₄″, when present, are chosen from hydrogen, C₁-C₆alkyl, amino(C₁-C₆)alkyl, and (C₁-C₆)haloalkyl.

Other embodiments of the invention provide compounds and salts of Formula V1 and Formula VII in which:

R₁ is chosen from C₁-C₁₀carbhydryl and (C₃-C₇cycloalkyl)C₀-C₄alkyl; each of which is substituted with one or more substituents independently chosen from halogen, hydroxy, amino, oxo, cyano, C₁-C₄alkoxy, and mono- and di-(C₁-C₄)alkylamino,

R₂ is chosen from hydrogen, halogen, C₁-C₆alkyl and C₁-C₄haloalkyl; and

R₃, R₃′, R₄, and R₄′, when present, are chosen from hydrogen, halogen, C₁-C₆alkyl, C₁-C₆alkoxy, (C₁-C₄)haloalkyl, (C₁-C₄)haloalkoxy, and mono- and di-C₁-C₄alkylamino; and

R₃″ and R₄″, when present, are hydrogen. In these embodiments Ar carries the definition set forth for compounds of Formula I or more preferably for compounds of Formula IA.

Still other embodiments of the invention provide to compounds and salts of Formula VI and Formula VII in which:

Ar is phenyl, pyridyl, or pyrimidinyl, each of which is mono-, di-, or tri-substituted with R_{A} (which carries the definition set forth for compounds and salts of Formula IA);

R₁ is chosen from C₁-C₁₀carbhydryl and (C₃-C₇cycloalkyl)C₀-C₄alkyl; each of which is substituted with 0 or more substituents independently chosen from halogen, hydroxy, amino, oxo, cyano, C₁-C₄alkoxy, and mono- and di-(C₁-C₄)alkylamino,

R₂ is chosen from hydrogen, halogen, C₁-C₆alkyl and (C₁-C₄)haloalkyl; and

R₃, R₃′, R₄, and R₄′, when present, are chosen from hydrogen, halogen, C₁-C₆alkyl, C₁-C₆alkoxy, (C₁-C₄)haloalkyl, (C₁-C₄)haloalkoxy, and mono- and di-C₁-C₄alkylamino.

R₃″ and R₄″ when present are hydrogen.

The invention also provides compounds and salts of Formula V1 and Formula VII in which:

Ar is chosen from phenyl, pyridyl, and pyrimidinyl, each of which is mono-, di-, or tri-substituted by substituents independently chosen from:

i) halogen, cyano, (C₁-C₄)haloalkyl, (C₁-C₄)haloalkoxy, hydroxy, amino, C₃-C₇cycloalkyl(C₀-C₄alkyl), mono- and di-C₁-C₆alkylamino, —CHO, and —C(═O)CH₃,

ii) C₁-C₆alkoxy and C₁-C₆alkyl which are substituted with 0 or 2 groups independently selected from halogen, hydroxy, cyano, amino, C₁-C₄alkyl, C₁-C₄alkoxy, mono- and di-C₁-C₄alkylamino, (C₁-C₄)haloalkyl, (C₁-C₄)haloalkoxy, and C₂-C₅alkanoyl, and

iii) 3- to 7-membered carbocyclic and heterocyclic groups, each of which is substituted with 0 or more substituents independently selected from halogen, oxo, hydroxy, amino, C₁-C₄alkyl, C₁-C₄alkoxy, and mono- and di-(C₁-C₄)alkylamino;

wherein at least one position ortho to the point of attachment of Ar in Formula I is substituted;

R₁ is chosen from C₁-C₁₀carbhydryl and (C₃-C₇cycloalkyl)C₀-C₄alkyl; each of which is substituted with one or more substituents independently chosen from halogen, hydroxy, amino, oxo, cyano, C₁-C₄alkoxy, and mono- and di-(C₁-C₄)alkylamino,

R₂ is chosen from hydrogen, halogen, C₁-C₆alkyl and (C₁-C₄)haloalkyl;

R₃, R₃′, R₄, and R₄′, when present, are chosen hydrogen, halogen, C₁-C₆alkyl, C₁-C₆alkoxy, (C₁-C₄)haloalkyl, (C₁-C₄)haloalkoxy, mono- and di-C₁-C₄alkylamino; and

R₃″ and R₄″, when present, are hydrogen.

In certain other embodiments, the invention provides compounds and salts of Formula VI and Formula VII in wherein:

Ar is phenyl, pyridyl, or pyrimidinyl, each of which is mono-, di-, or tri-substituted with substituents independently chosen from:

i) halogen, cyano, (C₁-C₄)haloalkyl, (C₁-C₄)haloalkoxy, hydroxy, amino, C₃-C₇cycloalkylC₀-C₄alkyl, mono- and di-(C₁-C₆)alkylamino, —CHO, and —C(═O)CH₃,

ii) C₁-C₆alkoxy and C₁-C₆alkyl which are substituted with from 0 to 2 groups independently selected from halogen, hydroxy, cyano, amino, C₁-C₄alkyl, C₁-C₄alkoxy, mono- and di-(C₁-C₄)alkylamino, (C₁-C₄)haloalkyl, (C₁-C₄)haloalkoxy, and C₂-C₅alkanoyl, and

iii) phenyl, pyridyl, pyrimidinyl, pyrazinyl, morpholinyl, piperidinyl, piperazinyl, and dioxolanyl, each of which is substituted with 0 or more substituents independently selected from halogen, oxo, hydroxy, amino, C₁-C₄alkyl, C₁-C₄alkoxy, and mono- and di-(C₁-C₄)alkylamino;

wherein at least one position ortho to the point of attachment of Ar in Formula VI or VII is substituted; and

R₁ is chosen from C₁-C₁₀alkyl and (C₃-C₇cycloalkyl)C₀-C₄alkyl, each of which is substituted with 0 or more substituents independently chosen from halogen, hydroxy, amino, oxo, cyano, C₁-C₄alkoxy, and mono- and di-(C₁-C₄)alkylamino,

R₂ is chosen from hydrogen, halogen, C₁-C₆alkyl and (C₁-C₄)haloalkyl; and

R₃ and R₄, when present, are chosen hydrogen, halogen, C₁-C₆alkyl, C₁-C₆alkoxy, (C₁-C₄)haloalkyl, and (C₁-C₄)haloalkoxy; and

R₃″ and R₄″, when present, are hydrogen.

The invention further provides compounds and salts of Formula VI and Formula VII in which:

Ar is phenyl, pyridyl or pyrimidinyl, each of which is mono-, di-, or tri-substituted with substituents independently chosen from halogen, cyano, hydroxy, amino, C₁-C₆alkyl, C₁-C₆alkoxy, (C₁-C₂)haloalkyl, (C₁-C₂)haloalkoxy, C₃-C₇cycloalkylC₀-C₂alkyl, mono- and di-(C₁-C₄)alkylamino, —CHO, and —C(═O)CH₃;

R₁ is chosen from C₁-C₈alkyl and (C₃-C₇cycloalkyl)C₀-C₂alkyl; each of which is substituted with from 0 to 3 substituents independently chosen from halogen, hydroxy, amino, oxo, cyano, C₁-C₄alkoxy, and mono- and di-(C₁-C₄)alkylamino,

R₂ is chosen from hydrogen, halogen, C₁-C₂alkyl and (C₁-C₂)haloalkyl; and

R₃, R₃′, R₄, and R₄′, when present, are chosen hydrogen, halogen, C₁-C₆alkyl, C₁-C₆alkoxy, (C₁-C₂)haloalkyl, and (C₁-C₂)haloalkoxy; and

R₃″ and R₄″, when present, are hydrogen.

In other embodiments the invention provides compounds and salts of Formula VI and Formula VII in which:

Ar is phenyl, pyridyl or pyrimidinyl, each of which is mono-, di-, or tri-substituted with substituents independently chosen from halogen, cyano, hydroxy, amino, C₁-C₄alkyl, C₁-C₃alkoxy, (C₁-C₂)haloalkyl, (C₁-C₂)haloalkoxy, and mono- and di-(C₁-C₄)alkylamino;

R₁ is chosen from C₁-C₈alkyl which is substituted with from 0 to 3 substituents independently chosen from halogen, hydroxy, amino, oxo, cyano, C₁-C₄alkoxy, and mono- and di-(C₁-C₄)alkylamino,

R₂ is chosen from hydrogen, halogen, C₁-C₂alkyl and (C₁-C₂)haloalkyl; and

R₃, R₃′, R₄, and R₄′, when present, are chosen hydrogen, halogen, C₁-C₄alkyl, C₁-C₄alkoxy, (C₁-C₂)haloalkyl, and (C₁-C₂)haloalkoxy.

Still other embodiments of the invention provide compounds and salts of Formula VI and Formula VII in wherein:

Ar is phenyl, pyridyl or pyrimidinyl, each of which is mono-, di-, or tri-substituted with substituents independently chosen from halogen, amino, C₁-C₄alkyl, C₁-C₃alkoxy, (C₁-C₂)haloalkyl, (C₁-C₂)haloalkoxy, and mono- and di-(C₁-C₂)alkylamino;

R₁ is chosen from C₁-C₈alkyl which is substituted with from 0 to 3 substituents independently chosen from halogen, hydroxy, amino, C₁-C₄alkoxy, and mono- and di-(C₁-C₄)alkylamino;

R₂ is chosen from hydrogen, methyl, and ethyl;

R₃, R₃′, R₄′, and R₄′, when present, are chosen hydrogen, halogen, ethyl, methyl, propyl, methoxy, ethoxy, and trifluoromethyl; and R₃″ and R₄″ are independently hydrogen or methyl.

In another aspect, the invention provides compounds of Formula VIII and the pharmaceutically acceptable salts thereof.

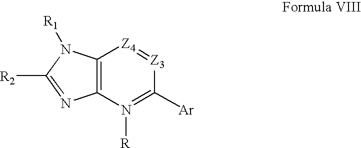

wherein

Ar is chosen from: phenyl which is mono-, di-, or tri-substituted, 1-naphthyl and 2-naphthyl, each of which is optionally mono-, di-, or tri-substituted, and optionally mono-, di-, or tri-substituted heteroaryl, having from 1 to 3 rings, 5 to 7 ring members in each ring and, in at least one ring, from 1 to about 3 heteroatoms selected from N, O, and S;

R is oxygen, methyl, or absent.

R₁ is chosen from —NR^{x}R_{y}, —C(O)NRₓR_{y}, —NRₓS(O)ₘR_{y}, —C(O)R^{x}, S(O)ₘRₓ, or CRₓR_{z}R_{z};

m is 0, 1, or 2;

Rₓ and R_{y} are independently selected from hydrogen, optionally substituted alkyl, optionally substituted alkenyl, optionally substituted alkynyl, optionally substituted cycloalkyl, optionally substituted (cycloalkyl)alkyl, optionally substituted mono- or di-alkylamino, optionally substituted alkanoyl, optionally substituted aryl, and optionally substituted heteroaryl, having from 1 to 3 rings, 5 to 7 ring members in each ring and, in at least one ring, from 1 to about 3 heteroatoms selected from N, O, and S;

R₂ is chosen from hydrogen, halogen, hydroxy, amino, cyano, nitro, alkyl, alkoxy, haloalkyl, haloalkoxy, aminoalkyl, and mono- and dialkylamino.

Z₃ is nitrogen or CR₃; Z₄ is nitrogen or CR₄; and Z₃ and Z are not both nitrogen.

R₃, R₄, and R_{z} are independently chosen from hydrogen, halogen, hydroxy, amino, cyano, nitro, optionally substituted alkyl, optionally substituted alkenyl, optionally substituted alkynyl, optionally substituted alkoxy, optionally substituted mono- and di-alkylamino, optionally substituted (cycloalkyl)alkyl, optionally substituted (cycloalkyl)oxy, optionally substituted (cycloalkyl)alkoxy, optionally substituted alkylthio, optionally substituted alkylsulfinyl, optionally substituted alkylsulfonyl, optionally substituted mono- and di-alkylcarboxamide, optionally substituted aryl, and optionally substituted heteroaryl, having from 1 to 3 rings, 5 to 7 ring members in each ring and, in at least one ring, from 1 to about 3 heteroatoms selected from the group consisting of N, O, and S.

Methods of Treatment

Compounds disclosed herein are useful in treating and preventing a variety of conditions including affective disorders, anxiety disorders, stress disorders, eating disorders, and drug addiction.

Affective disorders include all types of depression, bipolar disorder, cyclothymia, and dysthymia.

Anxiety disorders include generalized anxiety disorder, panic, phobias and obsessive-compulsive disorder.

Stress-related disorders include post-traumatic stress disorder, hemorrhagic stress, stress-induced psychotic episodes, psychosocial dwarfism, stress headaches, stress-induced immune systems disorders such as stress-induced fever, and stress-related sleep disorders.

Eating disorders include anorexia nervosa, bulimia nervosa, and obesity.

Modulators of CRF receptors are also useful in the treatment (e.g., symptomatic treatment) and prophylaxis of a variety of neurological disorders including supranuclear palsy, AIDS related dementias, multiinfarct dementia, neurodegenerative disorders such as Alzheimer's disease, Parkinson's disease, and Huntington's disease, head trauma, spinal cord trauma, ischemic neuronal damage, amyotrophic lateral sclerosis, disorders of pain perception such as fibromyalgia and epilepsy.

Additionally the compounds disclosed herein are useful as modulators of the CRF receptor in the treatment (e.g., symptomatic treatment) and prophylaxis of a number of gastrointestinal, cardiovascular, hormonal, autoimmune and inflammatory conditions. Such conditions include irritable bowel syndrome, ulcers, Crohn's disease, spastic colon, diarrhea, post operative ilius and colonic hypersensitivity associated with psychopathological disturbances or stress, hypertension, tachycardia, congestive heart failure, infertility, euthyroid sick syndrome, inflammatory conditions effected by rheumatoid arthritis and osteoarthritis, pain, asthma, psoriasis and allergies.

Compounds disclosed herein are also useful as modulators of the CRF1 receptor in the treatment and prophylaxis of animal disorders associated with aberrant CRF levels. These conditions include porcine stress syndrome, bovine shipping fever, equine paroxysmal fibrillation, and dysfunctions induced by confinement in chickens, sheering stress in sheep or human-animal interaction related stress in dogs, psychosocial dwarfism and hypoglycemia.

Typical subjects to which compounds disclosed herein may be administered will be mammals, particularly primates, especially humans. For veterinary applications, a wide variety of subjects will be suitable, e.g. livestock such as cattle, sheep, goats, cows, swine and the like; poultry such as chickens, ducks, geese, turkeys, and the like; and other domesticated animals particularly pets such as dogs and cats. For diagnostic or research applications, a wide variety of mammals will be suitable subjects including rodents (e.g. mice, rats, hamsters), rabbits, primates, and swine such as inbred pigs and the like.

Additionally, body fluids (e.g., blood, plasma, serum, CSF, lymph, cellular interstitial fluid, aqueous humor, saliva, synovial fluid, feces, or urine) and cell and tissue samples of the above subjects will be suitable substrates for use in in vitro applications, such as in vitro diagnostic and research applications.

The CRF binding compounds provided by this invention and labeled derivatives thereof are also useful as standards and reagents in determining the ability of test compounds (e.g., a potential pharmaceutical) to bind to a CRF receptor.

Labeled derivatives the CRF binding compounds provided by this invention are also useful as tracers for positron emission tomography (PET) imaging or for single photon emission computerized tomography (SPECT).

Compounds disclosed herein may be used for demonstrating the presence of CRF receptors in cell or tissue samples. This may be done by preparing a plurality of matched cell or tissue samples, at least one of which is prepared as an experiment sample and at least one of which is prepared as a control sample. The experimental sample is prepared by contacting (under conditions that permit binding of CRF to CRF receptors within cell and tissue samples) at least one of the matched cell or tissue samples that has not previously been contacted with any compound or salt disclosed herein with an experimental solution comprising the detectably-labeled preparation of the selected compound or salt at a first measured molar concentration. The control sample is prepared by in the same manner as the experimental sample and is incubated in a solution that contains the same ingredients as the experimental solution but that also contains an unlabelled preparation of the same compound or salt disclosed herein at a molar concentration that is greater than the first measured molar concentration.

The experimental and control samples are then washed to remove unbound detectably-labeled compound. The amount of detectably-labeled compound remaining bound to each sample is then measured and the amount of detectably-labeled compound in the experimental and control samples is compared. A comparison that indicates the detection of a greater amount of detectable label in the at least one washed experimental sample than is detected in any of the at least one washed control samples demonstrates the presence of CRF receptors in that experimental sample.

The detectably-labeled compound used in this procedure may be labeled with any detectable label, such as a radioactive label, a biological tag such as biotin (which can be detected by binding to detectably-labeled avidin), an enzyme (e.g., alkaline phosphatase, beta galactosidase, or a like enzyme that can be detected its activity, e.g., in a colorimetric assay) or a directly or indirectly luminescent label. When tissue sections are used in this procedure and the detectably-labeled compound is radiolabeled, the bound, labeled compound may be detected autoradiographically to generate an autoradiogram. When autoradiography is used, the amount of detectable label in an experimental or control sample may be measured by viewing the autoradiograms and comparing the exposure density of the autoradiograms.

The present invention also provides methods of inhibiting the binding of CRF to CRF receptors (preferably CFR1 receptors) which methods involve contacting a solution containing a CRF antagonist compound disclosed herein with cells expressing-CRF receptors, wherein the compound is present in the solution at a concentration sufficient to inhibit CRF binding to CRF receptors in vitro. This method includes inhibiting the binding of CRF to CRF receptors in vivo, e.g., in a patient given an amount of a compound of Formula I that would be sufficient to inhibit the binding of CRF to CRF receptors in vitro. In one embodiment, such methods are useful in treating physiological disorders associated with excess concentrations of CRF. The amount of a compound that would be sufficient to inhibit the binding of a CRF to the CRF receptor may be readily determined via a CRF receptor binding assay (see, e.g., Example 8), or from the EC₅₀ of a CRF receptor functional assay, such as a standard assay of CRF receptor mediated chemotaxis. The CRF receptors used to determine in vitro binding may be obtained from a variety of sources, for example from cells that naturally express CRF receptors, e.g. IMR32 cells or from cells expressing cloned human CRF receptors.

The present invention also provides methods for altering the activity of CRF receptors, said method comprising exposing cells expressing such receptors to an effective amount of a compound disclosed herein, wherein the compound is present in the solution at a concentration sufficient to specifically alter the signal transduction activity in response to CRF in cells expressing CRF receptors in vitro, preferred cells for this purpose are those that express high levels of CRF receptors (i.e., equal to or greater than the number of CRF1 receptors per cell found in differentiated IMR-32 human neuroblastoma cells), with IMR-32 cells being particularly preferred for testing the concentration of a compound required to alter the activity of CRF1 receptors. This method includes altering the signal transduction activity of CRF receptors in vivo, e.g., in a patient given an amount of a compound of Formula I that would be sufficient to alter the signal transduction activity in response to CRF in cells expressing CRF receptors in vitro. The amount of a compound that would be sufficient to alter the signal transduction activity in response to CRF of CRF receptors may also be determined via an assay of CRF receptor mediated signal transduction, such as an assay wherein the binding of CRF to a cell surface CRF receptor effects a changes in reporter gene expression.

The present invention also provides packaged pharmaceutical compositions for treating or preventing disorders responsive to CRF receptor modulation, e.g., eating disorders, depression or stress. The packaged pharmaceutical compositions include a container holding a therapeutically effective amount of at least one CRF1 receptor modulator as described supra and instructions for using the treating disorder responsive to CRF1 receptor modulation in the patient.

Pharmaceutical Preparations

The compounds of general Formula I may be administered orally, topically, transdermally, parenterally, by inhalation or spray or rectally or vaginally in dosage unit formulations containing conventional non-toxic pharmaceutically acceptable carriers, adjuvants and vehicles. The term parenteral as used herein includes subcutaneous, intravenous, intramuscular, intrathecal and like types of injection or infusion techniques. In addition, there is provided a pharmaceutical formulation comprising a compound provided herein and a pharmaceutically acceptable carrier. One or more compounds of general Formula I may be present in association with one or more non-toxic pharmaceutically acceptable carriers and/or diluents and/or adjuvants and if desired other active ingredients. The pharmaceutical compositions containing compounds of general Formula I may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropylmethylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions disclosed herein may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation products of the said partial esters with ethylene oxide, for example polyoxyethylene sorbitan monoleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of general Formula I may also be administered in the form of suppositories, e.g., for rectal administration of the drug. These compositions can be prepared by mixing the drug with a suitable non-irritating excipient that is solid at ordinary temperatures but liquid at body temperature and will therefore melt in the body to release the drug. Such materials include cocoa butter and polyethylene glycols.

Compounds of general Formula I may be administered parenterally in a sterile medium. The drug, depending on the vehicle and concentration used, can either be suspended or dissolved in the vehicle. Advantageously, one or more adjuvants such as preservatives, buffering agents, or local anesthetics can also be present in the vehicle.

Dosage levels of the order of from about 0.05 mg to about 100 mg per kilogram of body weight per day are useful in the treatment of the above-indicated conditions, preferred dosages range from about 0.1 to about 30 mg per kg and more preferably from about 0.5 to about 5 mg per kg per subject per day. The amount of active ingredient that may be combined with the carrier materials to produce a single dosage form will vary depending upon the host treated and the particular mode of administration. Dosage unit forms will generally contain between from about 0.1 mg to about 750 mg of an active ingredient.

Frequency of dosage may also vary depending on the compound used and the particular disease treated. However, for treatment of most CNS and gastrointestinal disorders, a dosage regimen of four times daily, preferably three times daily, more preferably two times daily and most preferably once daily is contemplated. For the treatment of stress and depression a dosage regimen of 1 or 2 times daily is particularly preferred.

It will be understood, however, that the specific dose level for any particular patient will depend upon a variety of factors including the activity of the specific compound employed, the age, body weight, general health, sex, diet, time of administration, route of administration, and rate of excretion, drug combination (i.e. other drugs being used to treat the patient) and the severity of the particular disease undergoing therapy.

Preferred compounds disclosed herein will have certain desirable pharmacological, biopharmaceutical, and/or biological properties. Such properties include, but are not limited to oral bioavailability, such that the preferred oral dosage forms discussed above can provide therapeutically effective levels of the compound in vivo. Penetration of the blood brain barrier is necessary for most compounds used to treat CNS disorders, while low brain levels of compounds used to treat periphereal disorders are generally preferred.

Assays may be used to predict these desirable pharmacological, biopharmaceutical, and/or biological properties. Assays used to predict bioavailability include transport across human intestinal cell monolayers, including Caco-2 cell monolayers. Toxicity to cultured hepatocycles may be used to predict compound toxicity, with non-toxic compounds being preferred. Penetration of the blood brain barrier of a compound in humans may be predicted from the brain levels of the compound in laboratory animals given the compound, e.g., intravenously.

Percentage of serum protein binding may be predicted from albumin binding assays. Examples of such assays are described in a review by Oravcová, et al. (Journal of Chromatography B (1996) volume 677, pages 1-27). Preferred compounds exhibit reversible serum protein binding. Preferably this binding is less than 99%, more preferably less than 95%, even more preferably less than 90%, and most preferably less than 80%.

Frequency of administration is generally inversely proportional to the in vivo half-life of a compound. In vivo half-lives of compounds may be predicted from in vitro assays of microsomal half-life as described by Kuhnz and Gieschen (Drug Metabolism and Disposition, (1998) volume 26, pages 1120-1127). Preferred half lives are those allowing for a preferred frequency of administration.

As discussed above, preferred compounds disclosed herein exhibit good activity in standard in vitro CRF receptor binding assays, preferably the assay as specified in Example 8, which follows. References herein to “standard in vitro receptor binding assay” are intended to refer to that protocol as defined in Example 8, which follows. Generally preferred compounds disclosed herein have an IC₅₀ (half-maximal inhibitory concentration) of about 1 micromolar or less, still more preferably and IC₅₀ of about 100 nanomolar or less even more preferably an IC₅₀ of about 10 nanomolar or less or even 1 nanomolar or less in such a defined standard in vitro CRF receptor binding assay as exemplified by Example 8 which follows.

### EXAMPLES

### Preparation of Compounds

The compounds of the present invention can be prepared in a number of methods that are readily apparent to one skilled in the art of organic synthesis. The compounds of the present invention can be synthesized using the methods described below, together with synthetic methods known in the art of synthetic organic chemistry, or variations thereon as appreciated by those skilled in the art. Preferred methods include but are not limited to those methods described below. Preferred methods for the preparation of compounds of the present invention include, but are not limited to, those described in Schemes I and II. Those who are skilled in the art will recognize that the starting materials may be varied and additional steps employed to produce compounds encompassed by the present invention.

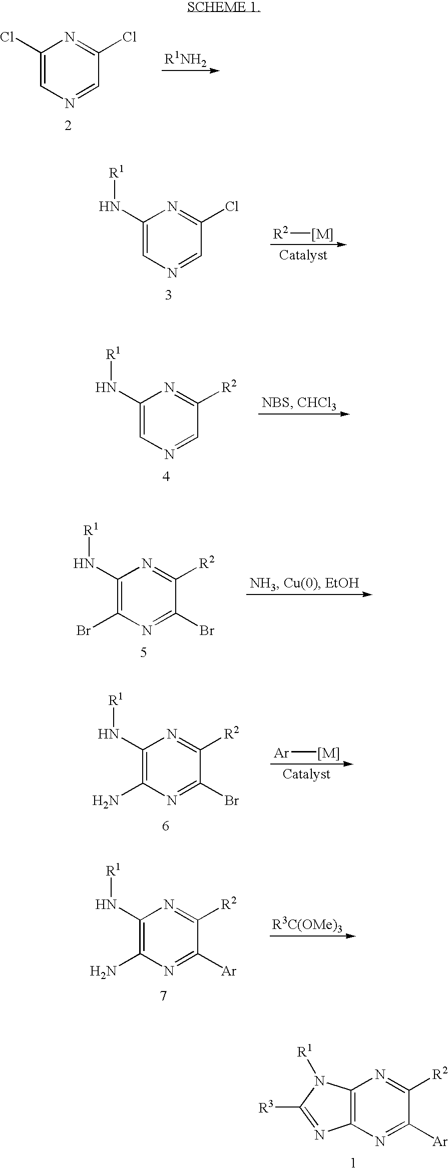

Commercially available 2,6-dichloropyrazine (2) can undergo monosubstitution with nucleophilic nitrogen compounds to give pyrazine 3. Thus, 2 may react with an amine in solvents such as, but not limited to, dichloromethane, acetonitrile, THF, DMF, N-methylpyrrolidinone, methyl sulfoxide, methanol, ethanol, and isopropanol at temperatures ranging from 0° C. to the boiling point of the solvent, with or without the presence of a suitable transition metal catalyst such as, but not limited to, palladium(II) acetate or tris(dibenzylideneacetone)dipalladium(0), a ligand such as, but not limited to, 1,1′-bis(diphenylphosphine)ferrocene, 2,2′-bis(diphenylphosphine)-1,1′-binaphthyl, dicyclohexyl(2-biphenyl)phosphine, tricyclohexylphosphine, or tri-tert-butylphosphine, and a base such as sodium or potassium tert-butoxide in inert solvents such as, but not limited to, toluene, ethyleneglycol dimethyl ether, diglyme, DMF, or N-methylpyrrolidinone at temperatures ranging from ambient to 100° C. Resulting monochloropyrazine 3 can be further converted to 4 by displacing the halogen atom with a variety of nucleophiles (R²-[M]), in the presence or absence of a transition metal catalyst. The aforementioned nucleophiles may include sodium or potassium (thio)alkoxide, alkylamine, and organometallic reagent such as, but not limited to, alkyl Grignard reagents, alkylboronic acids, esters or alkylboronic acids, or alkylstannanes. The aforementioned transition metal catalyst may be, but is not limited to, palladium(II) acetate or tris(dibenzylideneacetone)dipalladium(0), a ligand such as, but not limited to, 1,1′-bis(diphenylphosphine)ferrocene, 2,2′-bis(diphenylphosphine)-1,1′-binaphthyl, dicyclohexyl(2-biphenyl)phosphine, tricyclohexylphosphine, or tri-tert-butylphosphine, and a base such as sodium or potassium tert-butoxide in inert solvents such as, but not limited to, toluene, ethyleneglycol dimethyl ether, diglyme, DMF, or N-methylpyrrolidinone at temperatures ranging from ambient to 100° C. (Thio)alkoxypyrazines can be prepared by treating 3 with a sodium or potassium salt of an alcohol or thiol in an inert solvent such as THF, DMF, N-methylpyrrolidinone, or methyl sulfoxide at ambient temperature or at elevated temperature up to the boiling point of the solvent employed. Halogenation of 4 may be accomplished by a variety of methods known in the art, including treatment with N-chlorosuccinimide, bromine, N-bromosuccinimide, pyridinium tribromide, triphenylphosphine dibromide, iodine, and N-iodosuccinimide in solvents such as, but not limited to, dichloromethane, acetic acid, or methyl sulfoxide. The halogen atom on position ortho to the amino group in the pyrazine nucleus of compound 5 can be selectively displaced by an amine or (thio)alkoxide nucleophile. Thus, compound 6 can be prepared from 5 and ammonia in the presence of a suitable transition metal catalyst such as but not limited to copper (O), palladium(II) acetate or tris(dibenzylideneacetone)dipalladium(0), a ligand such as but not limited to 1,1′-bis(diphenylphosphine)ferrocene, 2,2′-bis(diphenylphosphine)-1,1′-binaphthyl, dicyclohexyl(2-biphenyl)phosphine, tricyclohexylphosphine, or tri-tert-butylphosphine, in inert solvents such as, but not limited to, ethanol, methanol, toluene, ethyleneglycol dimethyl ether, diglyme, DMF, or N-methylpyrrolidinone at temperatures ranging from ambient to 100° C. (Thio)alkoxypyrazines can be prepared by treating 5 with a sodium or potassium salt of an alcohol or thiol in an inert solvent such as THF, DMF, N-methylpyrrolidinone, or methyl sulfoxide at ambient temperature or at elevated temperature up to the boiling point of the solvent employed. Transition metal-catalyzed (hetero)aryl-aryl coupling of 6 can provide 7 by reaction with a metalloaryl reagent (Ar-[M]). More commonly employed reagent/catalyst pairs include aryl boronic acid/palladium(0) (Suzuki reaction; N. Miyaura and A. Suzuki, Chemical Reviews 1995, 95, 2457), aryl trialkylstannane/palladium(0) (Stille reaction; T. N. Mitchell, Synthesis 1992, 803), arylzinc/palladium(0) and aryl Grignard/nickel(II). Palladium(0) represents a catalytic system made of a various combinations of metal/ligand pairs which include, but are not limited to, tetrakis(triphenylphosphine)palladium(0), palladium(II) acetate/tri(o-tolyl)phosphine, tris(dibenzylideneacetone)dipalladium(0)/tri-tert-butylphosphine and dichloro[1,1′-bis(diphenylphosphine)ferrocene]palladium(0). Nickel(II) represents a nickel-containing catalyst such as [1,2-bis(diphenylphosphino)ethane]dichloronickel(II) and [1,3-bis(diphenylphosphino)propane]dichloronickel(II). Cyclization of the diaminopyrazine 7 to compound I can be accomplished by treatment with a number of reagents such as dimethoxymethylacetate or trialkylorthoesters such as triethylorthoformate or trimethylortoacetate in the presence or not of a suitable acidic catalyst such as, but not limited to, p-toluenesulfonic acid or sulfuric acid, in a solvent such as toluene, xylene, DMF, NMP, or methyl sulfoxide at temperatures ranging from 0° C. to 100° C. 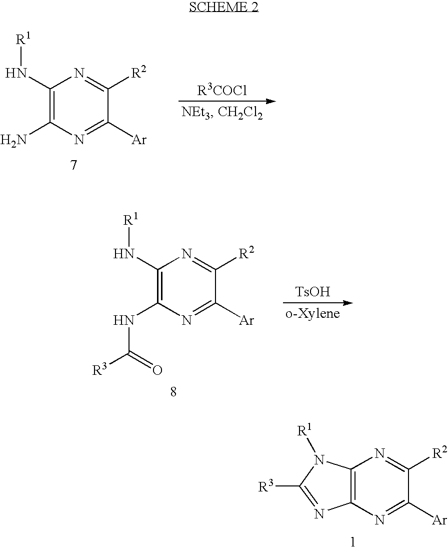
Compound 7 (obtained by the method illustrated in Scheme 1) can be acylated by treatment with one of a large number of acylating reagents such as but not limited to acyl chlorides, esters or carboxylic acids in the presence (or not) of a base such as triethylamine, pyridine or dimethylaniline in a solvent such as dichloromethane, chloroform, ether at temperatures between −78° C. and the boiling point of the solvent. Dehydration of amide 8 can be carried out by heating a with an inert solvent such as xylene, toluene, benzene, diphenylether, or DMSO in the presence (or not) of an acidic catalyst such as but not limited to p-toluenesulfonic acid, sulfuric acid, hydrochloric acid, at temperatures between 40° C. and 200° C.

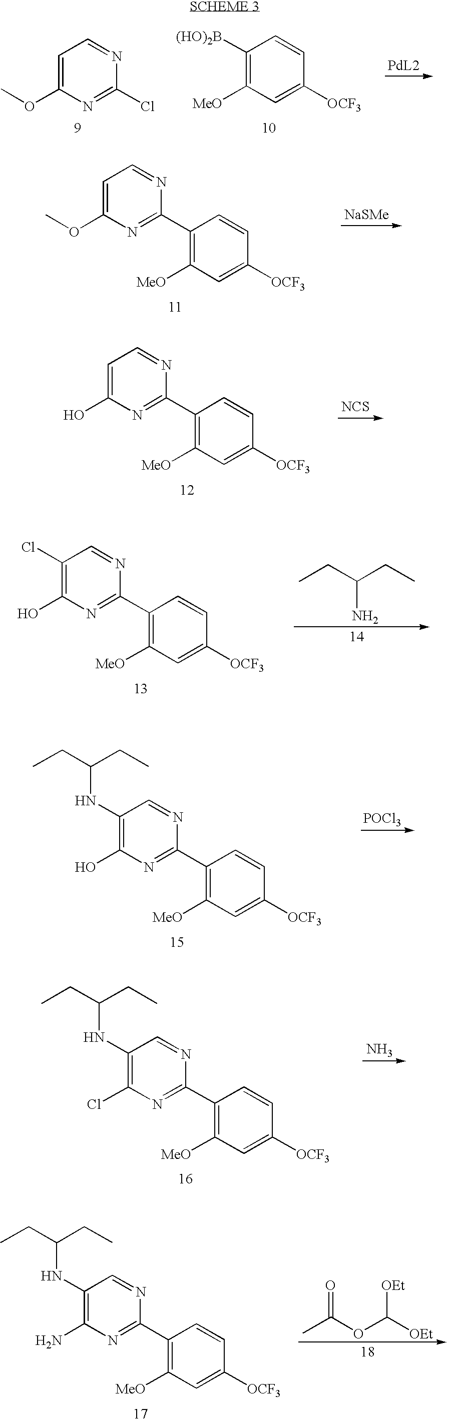

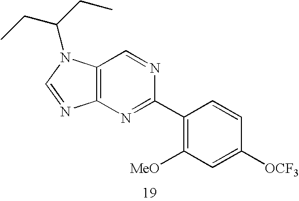

A mixture of chloropyrimide (9) and approximately 1 equivalent of boronic acid (10) is cross coupled with palladium catalyst to give 4-methoxy-2-(2-methoxy-4-methyl-phenyl)-pyrimidine (11). The coupling product (11) is dealkylated with sodium thiomethoxide or acid or base to give 4-hydroxy-2-(2-methoxy-4-methyl-phenyl)-pyrimidine (12). The latter compound is chlorinated with a reagent such as NCS to give 4-Hydroxy-5-chloro-2-(2-methoxy-4-methyl-phenyl)-pyrimidine (13), then treated with 1-ethylpropylamine (14), palladium catalyst and base, such as potassium tert-butoxide, to give 15. Compound 15 is chlorinated, or converted to the tosylate, then treated with a source of ammonia to afford the diamino compound 17. The diamine (17) is then cyclized with neat diethoxymethylacetate (18) to provide the imidazole 7-(1-ethyl-propyl)-2-(2-methoxy-4-trifluoromethoxy-phenyl) 7H-purine (19).

The preparation of the compounds of the present invention is illustrated further by the following examples, which are not to be construed as limiting the invention in scope or spirit to the specific procedures and compounds described in them.

Commercial reagents are used without further purification. Room or ambient temperature refers to 20 to 25° C. Concentration in vacuo implies the use of a rotary evaporator. TLC refers to thin layer chromatography. ¹H, ¹³C or ¹⁹F nuclear magnetic resonance spectra were obtained at 400 MHz, 100 MHz, or 376.3 MHz, respectively. Chemical shifts are expressed in the δ scale, using TMS as reference (δ=0 ppm). Mass spectral data were obtained either by Cl or APCI methods.

### Example 1

### Preparation of 1-(1-Ethyl-propyl)-5-(2-methoxy-4-trifluoromethoxy-phenyl)-6-methyl-1H-imidazo[4,5-b]pyrazine. (Compound 20) [Formula I: Ar=2-methoxy-4-trifluoromethoxyphenyl; R₁═CH(CH₂CH₃)₂; R₂═H; R₃═CH₃]

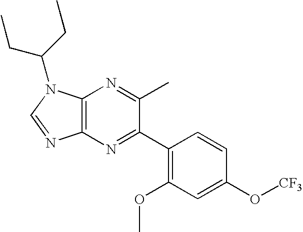

### Step 1: 6-Chloro-pyrazin-2-yl-(1-ethyl-propyl)-amine

A solution of 2,6-dichloropyrazine (2.2 g) and 1-ethylpropylamine (5 mL) in EtOH (10 mL) is heated at 140° C. in a Teflon-sealed pressure tube for 14 hours. The resulting solution is concentrated in vacuo, diluted by water, and extracted twice with hexane-ethyl ether. Combined extracts are dried (sodium sulfate), filtered, concentrated in vacuo, and the residue filtered through a short pad of silica gel. The filtrate is concentrated to yield 2-(3-pentylamino)-6-chloropyrazine as a brown oil that solidified on standing.

### Step 2: (1-Ethyl-propyl)-(6-methyl-pyrazin-2-yl)-amine

[1,3-bis(diphenylphosphino)propane]dichloronickel(II) (540 mg) is added to a solution of 6-chloro-pyrazin-2-yl-(1-ethyl-propyl)-amine (4.26 g, 21.3 mmol) in THF (30 mL) at room temperature. After 10 minutes at room temperature, methylmagnesium bromide (3.0 M in diethyl ether, 15.7 mL, 47.1 mmol) is added dropwise at 0° C. The reaction mixture is stirred at room temperature for 1 hour. The resulting dark solution is poured into aqueous ammonium chloride and extracted twice with ether. Combined extracts are dried (sodium sulfate), filtered, concentrated, and submitted to flash chromatography to yield the desired product as a light brown oil.

### Step 3. (3,5-Dibromo-6-methyl-pyrazin-2-yl)-(1-ethyl-propyl)-amine.

A solution of the oil obtained from Step 2 (3.7 g, 20.9 mmol) in chloroform (60 mL) is cooled to 0° C. (ice-water bath) and N-bromosuccinimide (7.8 g, 44.0 mmol) is added in portions. After the addition is complete, the reaction mixture is stirred for 1 hour more while being allowed to warm to room temperature. The mixture is then concentrated to a small volume in vacuo, triturated with hexane, filtered, washed with hexane, and the filtrate concentrated and submitted to flash chromatography on silica gel (8% ethyl acetate in hexane) to yield 3,5-dibromo-6-methyl-pyrazin-2-yl)-(1-ethyl-propyl)-amine.

### Step 4. 5-Bromo-N²-(1-ethyl-propyl)-6-methyl-pyrazine-2,3-diamine.

The product from step 3 (5.1 g, 15 mmol) is dissolved at room temperature in a solution of ammonia in ethanol (50 mL, 2M) in a pressure tube. Copper(0) (100 mg, 1.6 mmol) is added, and the mixture heated at 100 C for 16 hours. The reaction mixture is concentrated under reduced pressure, and the residue dissolved in ether and washed with brine (5×100 mL). The organic fractions are dried (magnesium sulfate), concentrated under reduced pressure, and the residue submitted to flash chromatography on silica gel eluting with ethyl acetate in hexanes, 5 to 15%). 5-Bromo-N²-(1-ethyl-propyl)-6-methyl-pyrazine-2,3-diamine is obtained as an oil. H-1 NMR: 4.2 (br, 2H), 3.94 (m, 1H), 3.83 (d, 1H), 2.39 (s, 3H), 1.63 (m, 2H), 1.49 (m, 2H), 0.91 (t, 6H).

### Step 5. N²-(3-Ethyl-propyl)-5-(2-methoxy-4-trifluoromethoxy-phenyl)-6-methyl-pyrazine-2,3-diamine

The product from step 4 (1.4 g, 5.1 mmol), 2-methoxy-4-trifluoromethoxyphenylboronic acid (2.4 g, 10 mmol), and tetrakis(triphenylphosphine)palladium(0) (100 mg) are suspended in a mixture of toluene (40 mL) and K₂CO₃ solution (10 mL, 2M in water) in a pressure tube. The reaction mixture is heated at 80° C. (oil bath temperature) for 16 h. After cooling, the heterogeneous mixture is partitioned between ether and sodium bicarbonate solution, and the organic phase washed with brine, dried (MgSO₄) and concentrated under reduced pressure. Flash chromatography (ethyl acetate 25% in hexanes) produces the title compound as a light-yellow solid. MS: 385 (M+1). H-1 NMR: 7.25 (d, 1H), 6.88 (d, 1H), 6.78 (s, 1H), 3.9-4.1 (m, 4H), 3.79 (s, 3H), 2.14 (s, 3H), 1.65 (m, 2H), 1.55 (m, 2H), 0.94 (t, 6H). C-13 NMR: 157.72, 140.80, 140.31, 143.78, 140.04, 132.88, 131.90, 127.77, 112.60, 104.39, 55.63, 52.63, 26.65, 20.83, 10.05. F-19 NMR: −58.08.

### Step 6. 1-(1-Ethyl-propyl)-5-(2-methoxy-4-trifluoromethoxy-phenyl)-6-methyl-1H-imidazo[4,5-b]pyrazine. (Compound 20)

The product from step 5 (150 mg, 0.39 mmol) is dissolved in dimethoxymethylacetate (3 mL) and the solution heated in a pressure tube submersed in an oil bath at 150° C. After 2 hours the reaction mixture is cooled to room temperature and the desired product obtained by preparative thin layer chromatography purification, eluting with ethyl acetate 30% in hexanes. MS: 395 (M+1). H-1 NMR: 8.21 (s, 1H), 7.36 (d, 1H), 6.94 (d, 1H), 6.82 (s, 1H), 4.41 (m, 1H), 3.77 (s, 3H), 2.47 (s, 3H), 2.04 (m, 4H), 0.84 (t, 6H). C-13 NMR: 157.75, 150.33, 147.74, 146.74, 146.35, 144.73, 138.76, 131.94, 127.62, 112.72, 104.28, 59.79, 55.65, 27.45, 22.22, 10.68. F-19 NMR: −58.07.

### Example 2

### Preparation of 1-(1-Ethyl-propyl)-5-(2-methoxy-4-trifluoromethoxy-phenyl)-2,6-dimethyl-1H-imidazo[4,5-b]pyrazine (Compound 21) [Formula I: Ar=2-methoxy-4-trifluoromethoxyphenyl; R₁═CH(CH₂CH₃)₂; R₂═CH₃; R₃═CH₃]

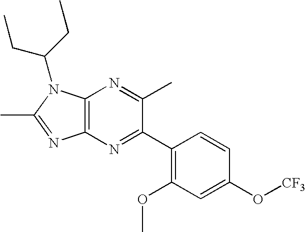

### Step 1: N-[3-(1-Ethyl-propylamino)-6-(2-methoxy-4-trifluoromethoxy-phenyl)-5-methyl-pyrazin-2-yl]-acetamide

N²-(1-Ethyl-propyl)-5-(2-methoxy-4-trifluoromethoxy-phenyl)-6-methyl-pyrazine-2,3-diamine (100 mg, 0.26 mmol, from example 1, step 5) is dissolved in dichloromethane (6 mL) containing trielthylamine (0.2 mL) under a nitrogen atmosphere (balloon) and taken to 0° C. (ice-water bath). Acetyl chloride (0.1 mL) is added dropwise, and the solution allowed to warm to room temperature overnight. The reaction mixture is diluted with ether and washed with sodium bicarbonate (sat sol, 2×50 mL) and brine (2×50 mL), dried (magnesium sulfate), concentrated under reduced pressure and loaded on a thin layer chromatography plate. Elution with ethyl acetate in hexanes (20%) affords the desired product. MS: 427 (M+1). H-1 NMR: 9.2 (br, 1H), 7.20 (d, 1H), 6.84 (d, 1H), 6.80 (s, 1H), 5.8 (br, 1H), 4.10 (m, 1H), 3.64 (s, 3H), 2.23 (s, 3H), 1.68 (m, 2H), 1.58 (m, 2H), 0.97 (t, 3H). F-19 NMR: −58.15.

### Step 2. 1-(1-Ethyl-propyl)-5-(2-methoxy-4-trifluoromethoxy-phenyl)-2,6-dimethyl-1H-imidazo[4.5-b]pyrazine. (Compound 21)

The product from step 1 (100 mg, 0.23 mmol) is dissolved in anhydrous o-xylene (3 mL) containing catalytic amounts of p-toluenesulfonic acid (15 mg). The mixture is heated in a pressure tube at 160° C. (oil bath temperature) for 6 h and then at 100° C. for 10 h. After cooling to room temperature, the solution is loaded on a thin layer chromatography plate, eluted with ethyl acetate in hexanes (30%). The desired product is obtained as a clear oil (40 mg, 43%). MS: 409 (M+1). H-1 NMR: 7.34 (d, 1H), 6.93 (d, 1H), 6.82 (d, 1H), 4.18 (m, 1H), 3.78 (s, 3H), 2.68 (s, 3H), 2.42 (s, 3H), 2.40 (m, 2H), 2.03 (m, 2H), 0.82 (t, 6H). F-19 NMR: −58.05.

### Example 3

### Preparation of 1-(1-Ethyl-propyl)-5-(2-methoxy-4-trifluoromethoxy-phenyl)-6-methyl-1H-imidazo[4,5-b]pyridine (Compound 24)

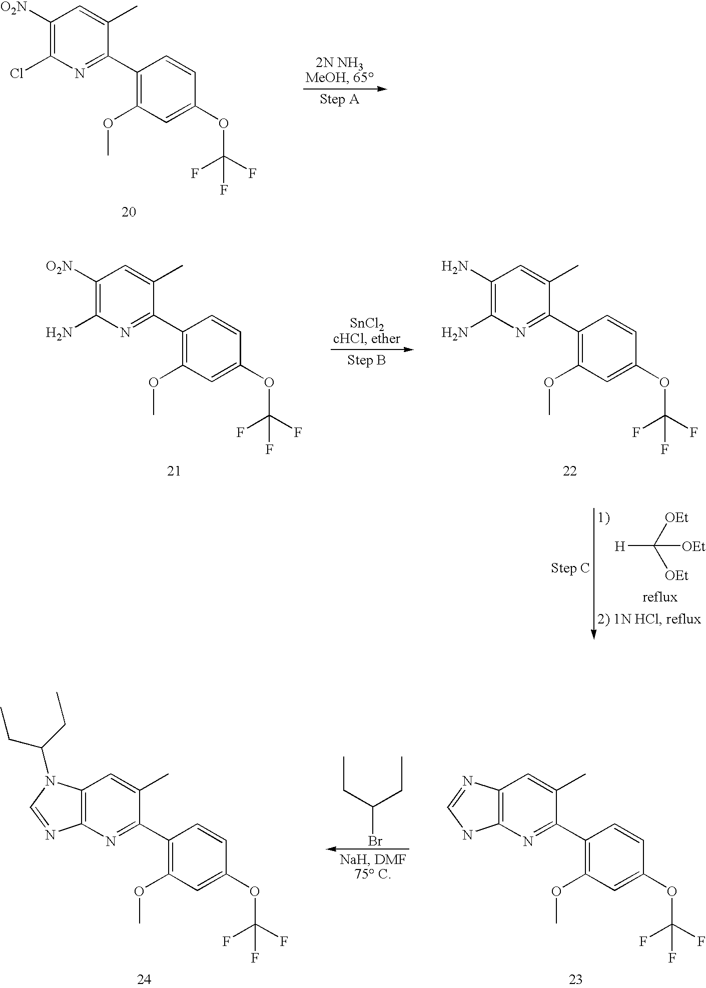

### Step A. Preparation of 6-(2-Methoxy-4-trifluoromethoxy-phenyl)-5-methyl-3-nitro-pyridin-2-ylamine

2 N ammonia solution in methanol (80 ml) is added to 2-chloro-6-(2-methoxy-4-trifluoromethoxy-phenyl)-5-methyl-3-nitro-pyridine (20) (5 g, 13.8 mmol) at room temperature. The mixture is stirred at 65° C. using a sealed tube for 1 week. After cooling to room temperature, yellow crystals formed are collected by filtration to obtain 6-(2-methoxy-4-trifluoromethoxy-phenyl)-5-methyl-3-nitro-pyridin-2-ylamine (21). Rf (hexane/EtOAc=4:1)=0.28.

### Step B. Preparation of 6-(2-methoxy-4-trifluoromethoxy-phenyl)-5-methyl-pyridine-2,3-diamine

6-(2-Methoxy-4-trifluoromethoxy-phenyl)-5-methyl-3-nitro-pyridin-2-ylamine (3 g, 8.7 mmol) in ether (50 ml) is added at 0° C. over a 10 minute period to a stirred solution of SnCl₂.H₂O (7.9 g, 35 mmol) in concentrated HCl (30 ml). The mixture is stirred at room temperature for 30 minutes and basified by aqueous NaOH solution. The mixture is extracted with EtOAc. The extract is dried over MgSO₄ and concentrated under reduced pressure to give 6-(2-methoxy-4-trifluoromethoxy-phenyl)-5-methyl-pyridine-2,3-diamine (23) as amorphous solid. Rf (CH₂Cl₂/MeOH=9:1)=0.26.

### Step C. Preparation of 5-(2-methoxy-4-trifluoromethoxy-phenyl)-6-methyl-3H-imidazo[4,5-b]pyridine

Triethyl orthoformate (25 ml) is added to 6-(2-methoxy-4-trifluoromethoxy-phenyl)-5-methyl-pyridine-2,3-diamine (2.75 g, 8.8 mmol) at room temperature. The mixture is refluxed for 5.5 hours and concentrated under reduced pressure. EtOH (10 ml) and 1N HCl (20 ml) are added to the residue and the mixture is refluxed for 1 hour. After cooling to room temperature, the solvent is removed under reduced pressure. Aqueous saturated NaHCO₃ solution is added to the residue and the mixture is extracted with EtOAc. The extract is dried over Na₂SO₄ and concentrated under reduced pressure. The crude product is purified by titration from ether-hexane to give 5-(2-methoxy-4-trifluoromethoxy-phenyl)-6-methyl-3H-imidazo[4,5-b]pyridine (23) as white solid. MS m/z 324.3 (M+H)⁺.

### Step D. Preparation of 1-(1-Ethyl-propyl)-5-(2-methoxy-4-trifluoromethoxy-phenyl)-6-methyl-1H-imidazo[4,5-b]pyridine (24)

NaH (37 mg, 0.93 mmol) is added to a solution of 5-(2-methoxy-4-trifluoromethoxy-phenyl)-6-methyl-3H-imidazo[4,5-b]pyridine (0.15 g, 0.46 mmol) in DMF (2 ml) at room temperature. After stirring for 20 minutes, 3-bromopentane (0.12 ml, 0.93 mmol) is added and the resulting mixture is stirred at 75° C. for 2 days. The mixture is poured into water (50 ml) and extracted with EtOAc. The extract is washed with brine and dried over Na₂SO₄. After removal of the solvent under reduced pressure, the residue is purified by preparative TLC to give the title compound (24) as colorless solid. Rf (CH₂Cl₂/MeOH=9:1)=0.17. MS m/z 395.3 (M+H)⁺.

### Example 4

### 5-(2,4-Dimethoxy-pyrimidin-5-yl)-1-(1-ethyl-propyl)-6-methyl-1H-imidazo[4,5-b]pyrazine

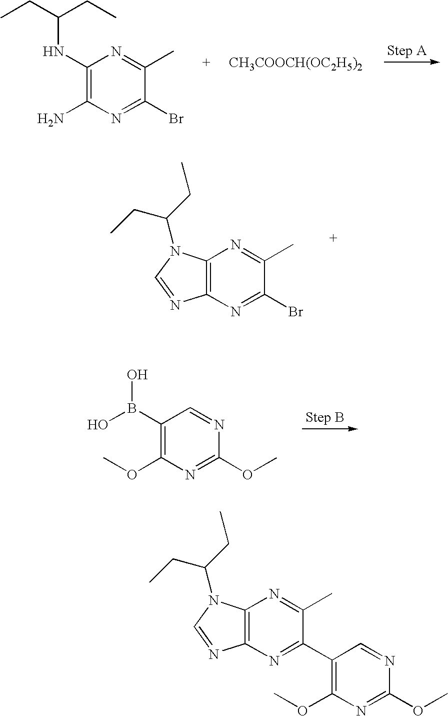

### Step A: 1-(1-Ethyl-propyl)-5,6-dimethyl-1H-imidazo[4,5-b]pyrazine:

N-(1-Ethyl-propyl)-5,6-dimethyl-pyrazine-2,3-diamine (274 mg, 1 mmol) is taken in 2 mL diethoxymethyl acetate and heated at 120° C. for 2 hours. TLC showed the reaction is complete. The reaction mixture is diluted with 10 ml ethyl acetate and washed with brine, dried with anhydrous Na₂SO₄. Purification by column with hexane/ethyl acetate(1:1) gives product as a brown oil, 282 mg, yield: 81%. ¹H NMR (δ, ppm, CDCl₃): 8.17 (1H, s, imidazole-H), 4.34 (1H, m, —NCH(Et)₂), 2.77 (3H, s, pyrazine-CH₃), 1.97-2.10 (4H, m, 2×-CH₂), 0.80 (6H, t, J=6.8 Hz, 2×-CH₃). MS (M+1): 283.1

### Step B: 5-(2,4-Dimethoxy-pyrimidin-5-yl)-1-(1-ethyl-propyl)-6-methyl-1H-imidazo[4,5-b]pyrazine:

1-(1-Ethyl-propyl)-5,6-dimethyl-1H-imidazo[4,5-b]pyrazine is taken in 6 ml toluene followed by the addition of Pd(PPh₃)₄ (100 mg), boronic acid (282 mg, 1.5 eq.) and Na₂SO₄ (1.0M in water, 2 ml). The resulting mixture is heated for 24 hours. The reaction mixture is extracted with ethyl acetate and dried with anhydrous Na₂SO₄. Purification by TLC gives product 170 mg, yield: 50%. ¹H NMR (δ, ppm, CDCl₃): 8.35 (1H, s, pyrimidine-H), 8.24 (1H, s, imidazole-H), 4.40 (1H, m, —NCH(Et)₂), 4.07 (3H, s, —OCH₃), 3.99 (3H, s, —OCH₃), 2.52 (3H, s, pyrazine-CH₃), 1.97-2.10 (4H, m, 2×-CH₂), 0.86 (6H, t, J=6.8 Hz, 2×-CH₃). MS (M+1): 343.3

### Example 5

### Additional Imidazo[4,5-b]Pyrazines

The following compounds, shown in Table I, (30-71) may be prepared using the methods given in reaction Schemes 1 and 2 and further illustrated in the preceding examples.

TABLE I

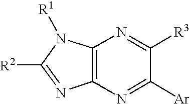

| Cmp # | R¹ | R² | R³ | Ar |
|---|---|---|---|---|
| 30 | 1-Ethyl-propyl | H | CH₃ | 4-Isopropyl-2,6-dimethoxyphenyl |
| 31 | 1-Ethyl-propyl | H | CH₃ | 4-(1-Fluoro-1-methyl-ethyl)-2,6- |
| | | | | dimethoxy-phenyl |
| 32 | 1-Ethyl-propyl | H | CH₃ | 4-(1-Hydroxy-1-methyl-ethyl)-2,6- |
| | | | | dimethoxy-phenyl |
| 33 | 1-Ethyl-propyl | H | CH₃ | 4-Acetyl-2,6-dimethoxy-phenyl |
| 34 | 1-Ethyl-propyl | H | CH₃ | 4-(1-Fluoro-ethyl)-2,6-dimethoxy- |
| | | | | phenyl |
| 35 | 1-Ethyl-propyl | H | CH₃ | 4-(1-Hydroxy-ethyl)-2,6-dimethoxy- |
| | | | | phenyl |
| 36 | 1-Ethyl-propyl | H | CH₃ | 4-Difluoromethyl-2,6-dimethoxy- |
| | | | | phenyl |
| 37 | 1-Ethyl-propyl | H | CH₃ | 4-Formyl-2,6-dimethoxy-phenyl |
| 38 | 1-Ethyl-propyl | H | CH₃ | 4-[1,3]Dioxolan-2-yl-2,6-dimethoxy- |
| | | | | phenyl |
| 39 | 1-Ethyl-propyl | H | CH₃ | 4-Isopropyl-2,6-dimethoxyphenyl |
| 40 | 1-Ethyl-propyl | H | CH₃ | 2-Methoxy-4,6-dimethylphenyl |
| 41 | 1-Ethyl-propyl | H | C₂H₅ | 4-Isopropyl-2,6-dimethoxyphenyl |
| 42 | 1-Ethyl-propyl | H | C₂H₅ | 4-(1-Fluoro-1-methyl-ethyl)-2,6- |
| | | | | dimethoxy-phenyl |
| 43 | 1-Ethyl-propyl | H | C₂H₅ | 4-(1-Hydroxy-1-methyl-ethyl)-2,6- |
| | | | | dimethoxy-phenyl |
| 44 | 1-Ethyl-propyl | H | C₂H₅ | 4-Acetyt-2,6-dimethoxy-phenyl |
| 45 | 1-Ethyl-propyl | H | C₂H₅ | 4-(1-Fluoro-ethyl)-2,6-dimethoxy- |
| | | | | phenyl |
| 46 | 1-Ethyl-propyl | H | C₂H₅ | 4-(1-Hydroxy-ethyl)-2,6-dimethoxy- |
| | | | | phenyl |
| 47 | 1-Ethyl-propyl | H | C₂H₅ | 4-Difluoromethyl-2,6-dimethoxy- |
| | | | | phenyl |
| 48 | 1-Ethyl-propyl | H | C₂H₅ | 4-Formyl-2,6-dimethoxy-phenyl |
| 49 | 1-Ethyl-propyl | H | C₂H₅ | 4-[1,3]Dioxolan-2-yl-2,6-dimethoxy- |
| | | | | phenyl |
| 50 | 1-Ethyl-propyl | H | C₂H₅ | 4-Isopropyl-2,6-dimethoxyphenyl |
| 51 | 1-Ethyl-propyl | H | C₂H₅ | 2-Methoxy-4,6-dimethylphenyl |
| 52 | 1-Ethyl-butyl | CH₃ | CH₃ | 4-Isopropyl-2,6-dimethoxyphenyl |
| 53 | 1-Ethyl-butyl | CH₃ | CH₃ | 4-(1-Fluoro-1-methyl-ethyl)-2,6- |
| | | | | dimethoxy-phenyl |
| 54 | 1-Ethyl-butyl | CH₃ | CH₃ | 4-(1-Hydroxy-1-methyl-ethyl)-2,6- |
| | | | | dimethoxy-phenyl |
| 55 | 1-Ethyl-butyl | CH₃ | CH₃ | 4-Acetyl-2,6-dimethoxy-phenyl- |
| 56 | 1-Ethyl-butyl | CH₃ | CH₃ | 4-(1-Fluoro-ethyl)-2,6-dimethoxy- |
| | | | | phenyl |
| 57 | 1-Ethyl-butyl | CH₃ | CH₃ | 4-(1-Hydroxy-ethyl)-2,6-dimethoxy- |
| | | | | phenyl |
| 58 | 1-Ethyl-butyl | F | CH₃ | 4-Difluoromethyl-2,6-dimethoxy- |
| | | | | phenyl |
| 59 | 1-Ethyl-butyl | F | CH₃ | 4-Formyl-2,6-dimethoxy-phenyl |
| 60 | 1-Ethyl-butyl | F | CH₃ | 4-[1,3]Dioxolan-2-yl-2,6-dimethoxy- |
| | | | | phenyl |
| 61 | 1-Ethyl-butyl | F | CH₃ | 4-Isopropyl-2,6-dimethoxyphenyl |
| 62 | 1-Ethyl-butyl | F | CH₃ | 2-Methoxy-4,6-dimethylphenyl |
| 63 | 1-Ethyl-butyl | H | CH₃ | 2-Methoxy-4- |
| | | | | trifluoromethoxyphenyl |
| 64 | 1-Ethyl-butyl | H | CH₃ | 2-Chloro-4-methoxyphenyl |
| 65 | 1-Ethyl-propyl | H | CH₃ | 2,4-Dichlorophenyl |
| 66 | 1-Ethyl-propyl | H | CH₃ | 2-Methoxy-4-isopropylphenyl |
| 67 | 1-Ethyl-butyl | H | CH₃ | 2-Methoxy-4-isopropylphenyl |
| 68 | 1-Propyl-butyl | H | CH₃ | 2-Methoxy-4-isopropylphenyl |
| 69 | 1-Cyclopropyl-propyl | H | CH₃ | 2-Methoxy-4-isopropylphenyl |
| 70 | 1-Dicyclopropylmethyl | H | CH₃ | 2-Methoxy-4-isopropylphenyl |
| 71 | 2-Methoxy-1- | H | CH₃ | 2-Methoxy-4-isopropylphenyl |
| | (methoxymethyl- | | | |
| | ethyl) | | | |
| 72 | 1-Ethyl-propyl | CF₃ | CH₃ | 2-Methoxy-4- |
| | | | | trifluoromethoxyphenyl |

### Example 6

The following compounds were prepared using the methods shown in above Schemes 1-3 and further illustrated by Examples 1-3. All compounds in Table II were tested in the radioligand binding assay (Example 8) and found to inhibit CRF binding to the human CRF1 receptors, exhibiting a Ki of 1 micromolar or less

TABLE 11

| Cmp.# | Structure | Molecular Name |
|---|---|---|
| 100 | 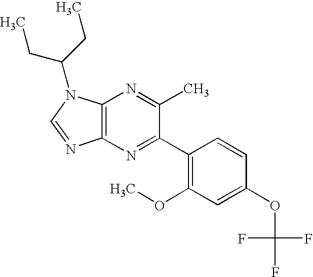 | 1-(1-Ethyl-propyl)-5-(2-methoxy-4- trifluoromethoxy-phenyl)-6-methyl-1H- imidazo[4,5-b]pyrazine |
| 101 | 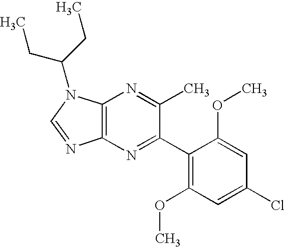 | 1-(1-Ethyl-propyl)-5-(2,6-dimethoxy-4- chloro-phenyl)-6-methyl-1H-imidazo[4,5- b]pyrazine |
| 102 | 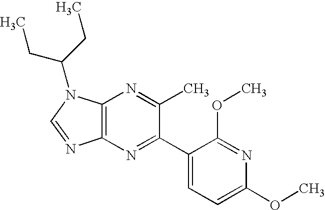 | 5-(2,6-Dimethoxy-pyridin-3-yl)-1-(1-ethyl- propyl)-6-methyl-1H-imidazo[4,5- b]pyrazine |
| 103 | 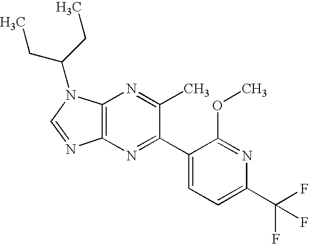 | 1-(1-Ethyl-propyl)-5-(2-methoxy-6- trifluoromethyl-pyridin-3-yl)-6-methyl-1H- imidazo[4,5-b]pyrazine |
| 104 | 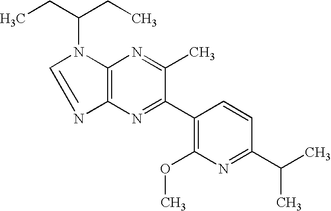 | 1-(1-Ethyl-propyl)-5-(2-methoxy-6- isopropyl-pyridin-3-yl)-6-methyl- 1H-imidazo[4,5-b]pyrazine |
| 105 | 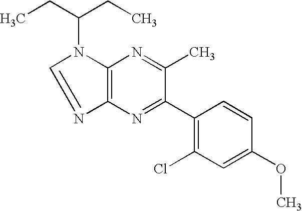 | 1-(1-Ethyl-propyl)-5-(2-chloro-4-methoxy- phenyl)-6-methyl-1H-imidazo[4,5- b]pyrazine |
| 106 | 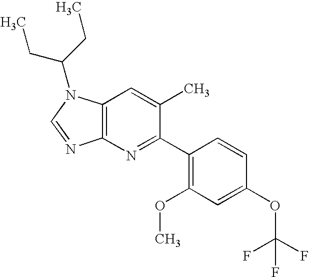 | 1-(1-Ethyl-propyl)-5-(2-methoxy-4- trifluoromethoxy-phenyl)-6-methyl-1H- imidazo[4,5-b]pyridine |
| 107 | 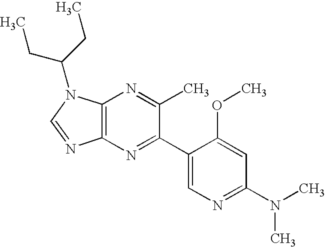 | {5-[1-(1-Ethyl-propyl)-6-methyl-1H- imidazo[4,5-b]pyrazin-5-yl]-4-methoxy- pyridin-2-yl}-dimethylamine |
| 108 | 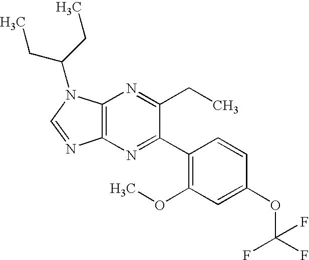 | 1-(1-Ethyl-propyl)-5-(2-methoxy-4- trifluoromethoxy-phenyl)-6-ethyl-1H- imidazo[4,5-b]pyrazine |
| 109 | 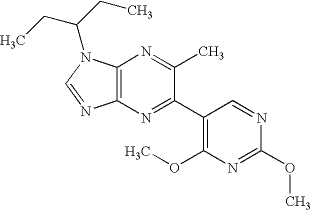 | 5-(2,4-Dimethoxy-pyrimidin-5-yl)-1- (1-ethyl-propyl)-6-methyl-1H-imidazo [4,5-b]pyrazine |
| 110 | 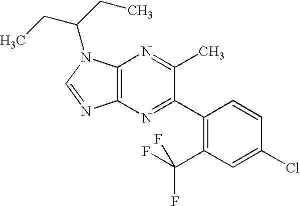 | 1-(1-Ethyl-propyl)-5-(2-trifluoromethyl-4- chloro-phenyl)-6-methyl-1H-imidazo[4,5- b]pyrazine |
| 111 | 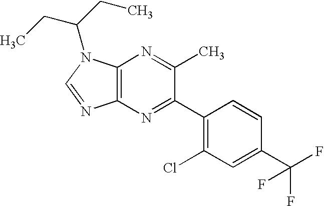 | 1-(1-Ethyl-propyl)-5-(2-chloro-4- trifluoromethyl-phenyl)-6-methyl-1H- imidazo[4,5-b]pyrazine |
| 112 | 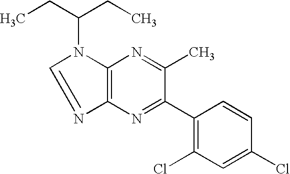 | 1-(1-Ethyl-propyl)-5-(2,4-dichloro-phenyl)- 6-methyl-1H-imidazo[4,5-b]pyrazine |
| 113 | 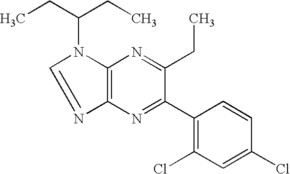 | 1-(1-Ethyl-propyl)-5-(2,4-dichloro-phenyl)- 6-ethyl-1H-imidazo[4,5-b]pyrazine |
| 114 | 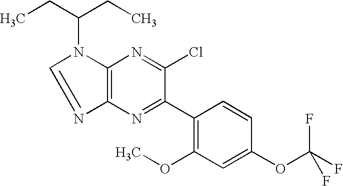 | 1-(1-Ethyl-propyl)-5-(2-methoxy-4- trifluoromethoxy-phenyl)-6-chloro-1H- imidazo[4,5-b]pyrazine |
| 115 | 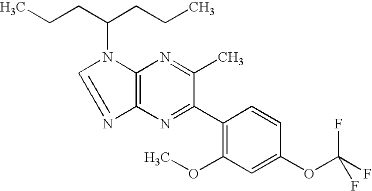 | 1-(1-propyl-butyl)-5-(2-methoxy-4- trifluoromethoxy-phenyl)-6-methyl-1H- imidazo[4,5-b]pyrazine |
| 116 | 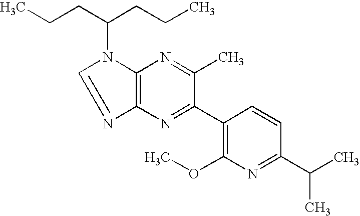 | 1-(1-Propyl-butyl)-5-(2-methoxy-6- isopropyl-pyridin-3-yl)-6-methyl-1H- imidazo[4,5-b]pyrazine |
| 117 | 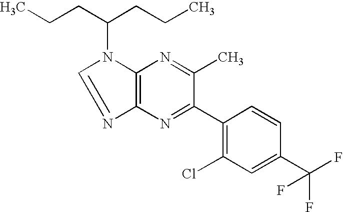 | 1-(1-Propyl-butyl)-5-(2-chloro-4- trifluoromethyl-phenyl)-6-methyl-1H- imidazo[4,5-b]pyrazine |
| 118 | 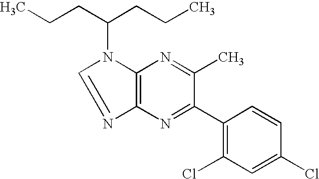 | 1-(1-Propyl-butyl)-5-(2,4-dichloro-phenyl)- 6-methyl-1H-imidazo[4,5-b]pyrazine |

| Cmp. | | MS |
|---|---|---|
| # | ¹H NMR (δ, ppm, CDCl₃) | (M + 1) |
| 104 | 8.21(1H, s, imidazole-H), 7.60(1H, d, J = 7.6 Hz, | 354.4 |
| | pyridine-H), 6.88(1H, d, J = 7.6 Hz, pyridine-H), | |
| | 4.42(1H, m, —NCH(Et)₂), 3.93(3H, s, —OCH₃), | |
| | 3.0(1H, m, —CHMe₂), 2.52(3H, s, pyrazine-CH₃), | |
| | 1.97-2.10(4H, m, 2X—CH₂), 1.32(6H, d, J = 6.8 Hz, | |
| | —CH(CH₃)₂, 0.87(6H, t, J = 7.2 Hz, 2X—CH₃ | |
| 105 | 8.24(1H, s, imidazole-H), 7.31(1H, d, J = 8.4 Hz, | 345.3 |
| | phenyl-H), 7.04(1H, d, J = 2.8 Hz, phenyl-H), | |
| | 6.93(1H, dd, J = 8.4 Hz, 2.4 Hz,, phenyl-H), | |
| | 4.43(1H, m, —NCH(Et)₂), 3.86(3H, s, —OCH₃), | |
| | 2.50(3H, s, pyrazine-CH₃), 1.97-2.10(4H, m, | |
| | 2X—CH₂), 0.87(6H, t, J = 7.6 Hz, 2X—CH₃) | |
| 108 | 8.22(1H, s, imidazole-H), 7.35(1H, d, J = 8.4 Hz, | 409.4 |
| | phenyl-H), 6.95(1H, dd, J = 8.4 Hz, 1.6 Hz,, | |
| | phenyl-H), 6.83(1H, d, J = 1.6 Hz, phenyl-H), | |
| | 4.41(1H, m, —NCH(Et)₂), 3.77(3H, s, —OCH₃), | |
| | 2.60-2.90(2H, m, pyrazine-CH₂Me), 1.97-2.20(4H, m, | |
| | 2X—CH₂), 1.22(3H, t, J = 7.6 Hz, | |
| | pyrazine-CH₂CH₃), 0.87(6H, t, J = 7.6 Hz, 2X—CH₃) | |
| 109 | 8.17(1H, s, imidazole-H), 4.34(1H, m, —NCH(Et)₂), | 343.3 |
| | 2.77(3H, s, pyrazine-CH₃), 1.97-2.10(4H, m, 2X— | |
| | CH₂), 0.80(6H, t, J = 6.8 Hz, 2X—CH₃) | |
| 110 | 8.27(1H, s, imidazole-H), 7.79(1H, s, phenyl-H), | 383.3 |
| | 7.62(1H, d, J = 8.4 Hz, phenyl-H), 7.33(1H, d, | |
| | J = 8.4 Hz, phenyl-H), 4.44(1H, m, —NCH(Et)₂), | |
| | 2.40(3H, s, pyrazine-CH₃), 1.97-2.10(4H, m, 2X— | |
| | CH₂), 0.87(6H, t, J = 7.6 Hz, 2X—CH₃) | |
| 111 | 8.29(1H, s, imidazole-H), 7.79(1H, s, phenyl-H), | 383.3 |
| | 7.66(1H, d, J = 8.0 Hz, phenyl-H), 7.55(1H, d, | |
| | J = 8.0 Hz, phenyl-H), 4.45(1H, m, —NCH(Et)₂), | |
| | 2.51(3H, s, pyrazine-CH₃), 1.97-2.10(4H, m, 2X— | |
| | CH₂), 0.88(6H, t, J = 7.6 Hz, 2X—CH₃) | |
| 112 | 8.27(1H, s, imidazole-H), 7.53(1H, s, phenyl-H), | 349.4 |
| | 7.30-7.40(2H, m, phenyl-H), 4.44(1H, m, | |
| | —NCH(Et)₂), 2.50(3H, s, pyrazine-CH₃), 1.97-2.10(4H, | |
| | m, 2X—CH₂), 0.88(6H, t, J = 7.6 Hz, 2X—CH₃) | |
| 113 | 8.27(1H, s, imidazole-H), 7.53(1H, s, phenyl-H), | 363.3 |
| | 7.30-7.40(2H, m, phenyl-H), 4.43(1H, m, | |
| | —NCH(Et)₂), 2.60-2.90(2H, s, pyrazine-CH₂), | |
| | 1.97-2.10(4H, m, 2X—CH₂), 1.20-1.30(3H, m, pyrazine- | |
| | CH₂CH₃), 0.88(6H, t, J = 7.6 Hz, 2X—CH₃) | |
| 114 | 8.31(1H, s, imidazole-H), 7.41(1H, d, J = 8.0 Hz, | 415.3 |
| | phenyl-H), 6.96(1H, d, J = 8.0 Hz, phenyl-H), | |
| | 6.85(1H, s, phenyl-H), 4.42(1H, m, —NCH(Et)₂), | |
| | 3.82(3H, s, —OCH₃), 1.97-2.10(4H, m, 2X—CH₂), | |
| | 0.89(6H, t, J = 7.6 Hz, 2X—CH₃) | |
| 115 | 8.23(1H, s, imidazole-H), 7.38(1H, d, J = 8.0 Hz, | 423.3 |
| | phenyl-H), 6.95(1H, d, J = 8.0 Hz, phenyl-H), | |
| | 6.84(1H, s, phenyl-H), 4.62(1H, m, —NCH(Et)₂), | |
| | 3.80(3H, s, —OCH₃), 2.49(3H, s, pyrazine-CH₃), | |
| | 1.84-2.10(4H, m, 2X—CH₂Et), 1.18-1.24(4H, m, | |
| | 2X—CH₂Me), 0.91(6H, t, J = 7.2 Hz, 2X—CH₃) | |
| 116 | 8.21(1H, s, imidazole-H), 7.60(1H, d, J = 7.2 Hz, | 382.3 |
| | pyridine-H), 6.88(1H, d, J = 7.2 Hz, pyridine-H), | |
| | 4.62(1H, m, —NCH(Et)₂), 3.84(3H, s, —OCH₃), | |
| | 3.10(1H, m, —CHMe₂), 2.53(3H, s, pyrazine-CH₃), | |
| | 2.00-2.10(2H, m, —CH₂Et), 1.95-1.98(2H, m, | |
| | —CH₂Et), 1.33(6H, d, J = 7.2 Hz, —CH(CH₃)₂), | |
| | 1.08-1.30(4H, m, 2X—CH₂Me), 0.91(6H, t, J = | |
| | 7.6 Hz, 2X—CH₃) | |
| 117 | 8.28(1H, s, imidazole-H), 7.78(1H, s, phenyl-H), | 411.4 |
| | 7.66(1H, d, J = 8.0 Hz, phenyl-H), 7.55(1H, d, | |
| | J = 8.0 Hz, phenyl-H), 4.64(1H, m, —NCH(Et)₂), | |
| | 2.50(3H, s, pyrazine-CH₃), 2.00-2.14(2H, m, | |
| | —CH₂Et), 1.90-2.00(2H, m, —CH₂Et), 1.18-1.35(4H, | |
| | m, 2X—CH₂Me), 0.92(6H, t, J = 7.6 Hz, 2X—CH₃) | |
| 118 | 8.26(1H, s, imidazole-H), 7.53(1H, s, phenyl-H), | 377.5 |
| | 7.34-7.38(2H, m, phenyl-H), 4.63(1H, m, | |
| | —NCH(Et)₂), 2.50(3H, s, pyrazine-CH₃), 2.00-2.10(2H, | |
| | m, —CH₂Et), 1.90-2.00(2H, m, —CH₂Et), | |
| | 1.18-1.35(4H, m, 2X—CH₂Me), 0.91(6H, t, J = | |
| | 7.2 Hz, 2X—CH₃) | |
| 72 | 7.36(1H, d, J = 8.0 Hz, phenyl-H), 6.97(1H, dd, | 463.4 |
| | J = 8.0 Hz, 2Hz,, phenyl-H), 6.85(1H, d, J = 2 | |
| | Hz, phenyl-H), 4.39(1H, m, —NCH(Et)₂), 3.80(3H, s, | |
| | —OCH₃), 2.51(3H, s, pyrazine-CH₃), 2.50-2.60(2H, m, | |
| | —CH₂), 1.97-2.10(2H, m, —CH₂), 0.84(6H, t, | |
| | J = 7.2 Hz, 2X—CH₃) | |

### Example 7

### Additional Compounds of Formula I

R₁-Matrix, Het-Matrix, and Ar-Matrix tables below set forth a number of which are prepared by the methods analogous to those shown in Reaction 3 above. Compounds are formed by combining any element from the R1 Matrix with any element from the Het-matrix to form an R1-Het moiety, and then combining this moiety with any element of the Ar-Matrix. For example, the combination of element 101 from the R1-Matrix, with element 208 from the Het-matrix, gives the moiety 101208. This moiety is then combined with element 304 from the Ar-matrix, to form compound 5101208304, which is 2-(2,4-dimethoxy-phenyl)-7-(1-ethyl-propyl)-6-methoxy-8-methyl-7H-purine.

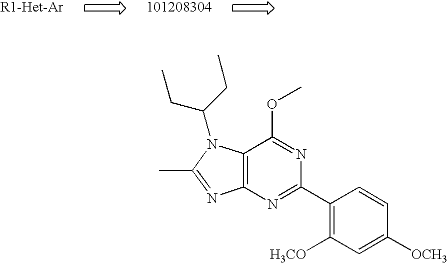

R1-Matrix

| | |
|---|---|
| 101 | 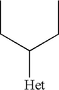 |
| 102 | 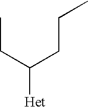 |
| 103 | 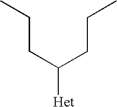 |
| 104 | 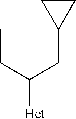 |
| 105 | 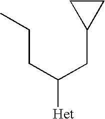 |
| 106 | 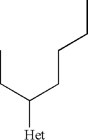 |
| 107 | 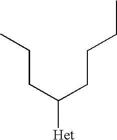 |
| 108 | 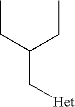 |
| 109 | 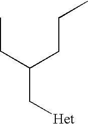 |
| 110 | 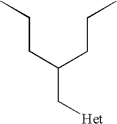 |
| 111 | 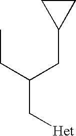 |
| 112 | 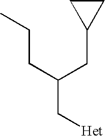 |
| 113 | 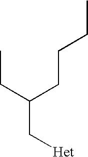 |
| 114 | 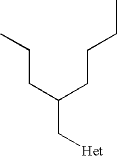 |
| 115 | |

Het1-Matrix

| | |
|---|---|
| 201 | 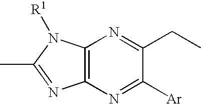 |
| 202 | 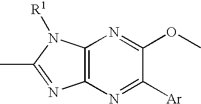 |
| 203 | 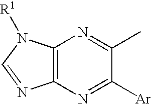 |
| 204 | 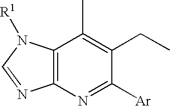 |
| 205 | 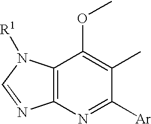 |
| 206 | 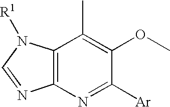 |
| 207 | 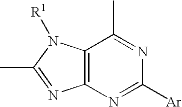 |
| 208 | 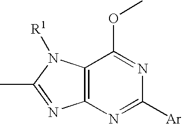 |
| 209 | 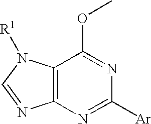 |
| 210 | 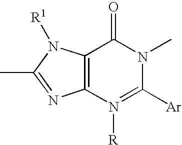 |
| 211 | 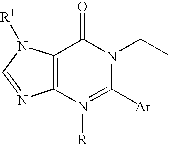 |
| 212 | 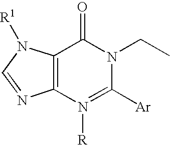 |
| 213 | 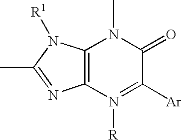 |
| 214 | 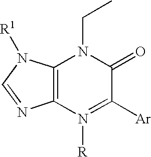 |
| 215 | 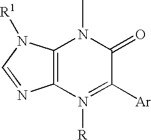 |

Ar-Matrix

| | |
|---|---|
| 301 | 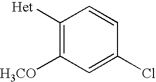 |
| 302 | 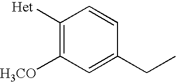 |
| 303 | 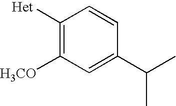 |
| 304 | 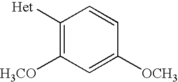 |
| 305 | 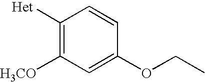 |
| 306 | 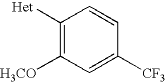 |
| 307 | 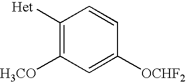 |
| 308 | 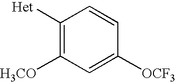 |
| 309 | 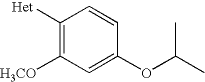 |
| 310 | 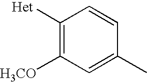 |
| 311 | 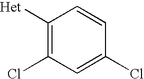 |
| 312 | 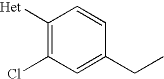 |
| 313 | 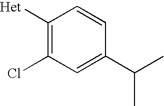 |
| 314 | 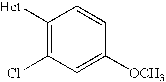 |
| 315 | 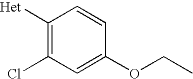 |
| 316 | 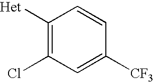 |
| 317 | 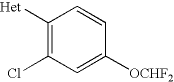 |
| 318 | 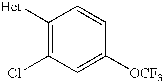 |
| 319 | 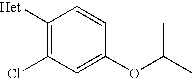 |
| 320 | 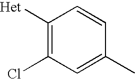 |
| 321 | 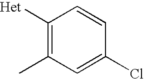 |
| 322 | 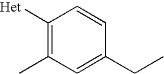 |
| 323 | 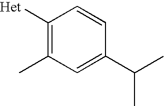 |
| 324 | 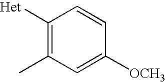 |
| 325 | 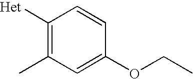 |
| 326 | 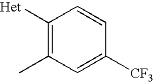 |
| 327 | 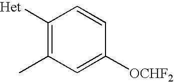 |
| 328 | 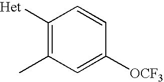 |
| 329 | 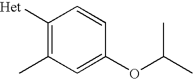 |
| 330 | 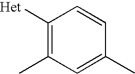 |
| 331 | 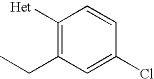 |
| 332 | 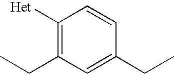 |
| 333 | 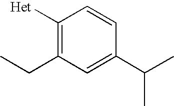 |
| 334 | 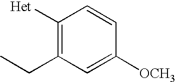 |
| 335 | 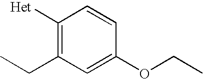 |
| 336 | 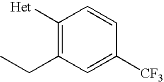 |
| 337 | 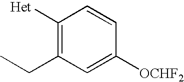 |
| 338 | 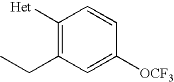 |
| 339 | 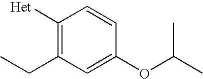 |
| 340 | 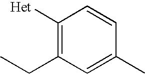 |
| 341 | 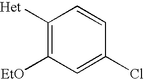 |
| 342 | 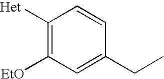 |
| 343 | 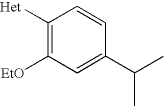 |
| 344 | 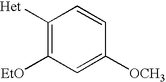 |
| 345 | 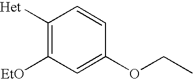 |
| 346 | 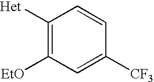 |
| 347 | 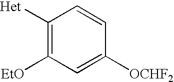 |
| 348 | 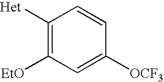 |
| 349 | 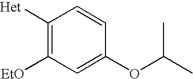 |
| 350 | 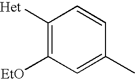 |
| 351 | 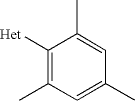 |
| 352 | 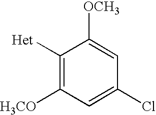 |
| 353 | 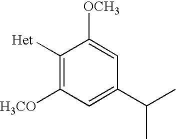 |
| 354 | 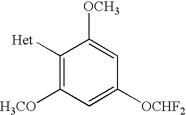 |
| 355 | 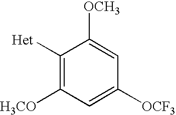 |
| 356 | 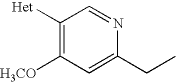 |
| 357 | 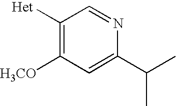 |
| 358 | 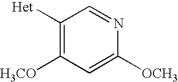 |
| 359 | 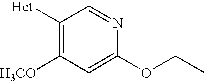 |
| 360 | 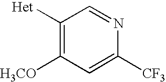 |
| 361 | 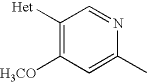 |
| 362 | 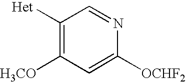 |
| 363 | 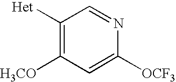 |
| 364 | 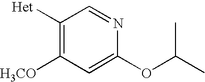 |
| 365 | 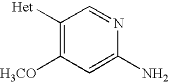 |
| 366 | 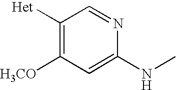 |
| 367 | 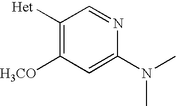 |
| 368 | 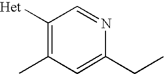 |
| 369 | 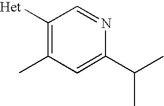 |
| 370 | 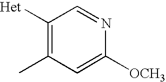 |
| 371 | 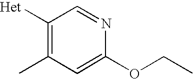 |
| 372 | 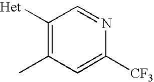 |
| 373 | 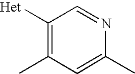 |
| 374 | 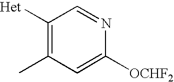 |
| 375 | 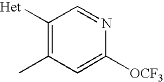 |
| 376 | 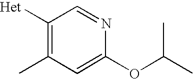 |
| 377 | 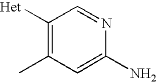 |
| 378 | 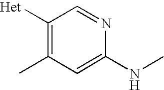 |
| 379 | 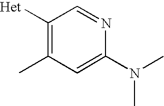 |
| 380 | 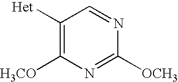 |

### Example 8

### Assay for CRF Receptor Binding Activity

As discussed above, the following assay is defined herein as a standard in vitro CRF receptor binding assay.

The pharmaceutical utility of compounds of this invention is indicated by the following assay for CRF1 receptor activity. The CRF receptor binding is performed using a modified version of the assay described by Grigoriadis and De Souza (Methods in Neurosciences, Vol. 5, 1991). IMR-32 human neuroblastoma cells, a cell-line that naturally expresses the CRF1 receptor, are grown in IMR-32 Medium, which consists of EMEM w/Earle's BSS (JRH Biosciences, Cat# 51411) plus, as supplements, 2 mM L-Glutamine, 10% Fetal Bovine Serum, 25 mM HEPES (pH 7.2), 1 mM Sodium Pyruvate and Non-Essential Amino Acids (JRH Biosciences, Cat# 58572). The cells are grown to confluence and split three times (all splits and harvest are carried out using NO-ZYME, JRH Biosciences, Cat# 59226). The cells are first split 1:2, incubated for 3 days and split 1:3, and finally incubated for 4 days and split 1:5. The cells are then incubated for an additional 4 days before being differentiated by treatment with 5-bromo-2′deoxyuridine (BrdU, Sigma, Cat# B9285). The medium is replaced every 34 days with IMR-32 medium w/2.5 μM BrdU and the cells are harvested after 10 days of BrdU treatment and washed with calcium and magnesium-free PBS.

To prepare receptor containing membranes cells are homogenized in wash buffer (50 mM Tris HCl, 10 mM MgCl₂, 2 mM EGTA, pH 7.4) and centrifuged at 48,000×g for 10 minutes at 4° C. The pellet is re-suspended in wash buffer and the homogenization and centrifugation steps are performed two additional times.

Membrane pellets (containing CRF receptors) are re-suspended in 50 mM Tris buffer pH 7.7 containing 10 mM MgCl₂ and 2 mM EDTA and centrifuged for 10 minutes at 48,000 g. Membranes are washed again and brought to a final concentration of 1500 μg/ml in binding buffer (Tris buffer above with 0.1% BSA, 15 mM bacitracin and 0.01 mg/ml aprotinin.). For the binding assay, 100 ul of the membrane preparation are added to 96 well microtube plates containing 100 μL of ¹²⁵I-CRF (SA 2200 Ci/mmol, final concentration of 100 μM) and 50 μL of test compound. Binding is carried out at room temperature for 2 hours. Plates are then harvested on a BRANDEL 96 well cell harvester and filters are counted for gamma emissions on a Wallac 1205 BETAPLATE liquid scintillation counter. Non-specific binding is defined by 1 mM cold CRF. IC₅₀ values are calculated with the non-linear curve fitting program RS/1 (BBN Software Products Corp., Cambridge, Mass.). The binding affinity for the compounds of Formula I expressed as IC₅₀ value, generally ranges from about 0.5 nanomolar to about 10 micromolar. Preferred compounds of Formula I exhibit IC₅₀ values of less than or equal to 1.5 micromolar, more preferred compounds of Formula I exhibit IC₅₀ values of less than 500 nanomolar, still more preferred compounds of Formula I exhibit IC₅₀ values of less than 100 nanomolar, and most preferred compound of Formula I exhibit IC₅₀ values of less than 10 nanomolar. The compounds shown in Examples 1-6 and Tables I and II have been tested in this assay and found to exhibit IC₅₀ values of less than or equal to 4 micromolar.

### Example 9

### Preparation of Radiolabeled Probe Compounds of the Invention

The compounds of the invention are prepared as radiolabeled probes by carrying out their synthesis using precursors comprising at least one atom that is a radioisotope. The radioisotope is preferably selected from of at least one of carbon (preferably ¹⁴C), hydrogen (preferably ³H), sulfur (preferably ³⁵S), or iodine (preferably ¹²⁵I). Such radiolabeled probes are conveniently synthesized by a radioisotope supplier specializing in custom synthesis of radiolabeled probe compounds. Such suppliers include Amersham Corporation, Arlington Heights, Ill.; Cambridge Isotope Laboratories, Inc. Andover, Mass.; SRI International, Menlo Park, Calif.; Wizard Laboratories, West Sacramento, Calif.; ChemSyn Laboratories, Lexena, Kans.; American Radiolabeled Chemicals, Inc., St. Louis, Mo.; and Moravek Biochemicals Inc., Brea, Calif.

Tritium labeled probe compounds are also conveniently prepared catalytically via platinum-catalyzed exchange in tritiated acetic acid, acid-catalyzed exchange in tritiated trifluoroacetic acid, or heterogeneous-catalyzed exchange with tritium gas. Such preparations are also conveniently carried out as a custom radiolabeling by any of the suppliers listed in the preceding paragraph using the compound disclosed herein as substrate. In addition, certain precursors may be subjected to tritium-halogen exchange with tritium gas, tritium gas reduction of unsaturated bonds, or reduction using sodium borotritide, as appropriate.

### Example 10

### Receptor Autoradiography

Receptor autoradiography (receptor mapping) is carried out in vitro as described by Kuhar in sections 8.1.1 to 8.1.9 of Current Protocols in Pharmacology (1998) John Wiley & Sons, New York, using radiolabeled compounds disclosed herein prepared as described in the preceding Examples.

### Example 11

### Additional Aspects of Preferred Compounds of the Invention

The most preferred compounds of the invention are suitable for pharmaceutical use in treating human patients. Accordingly, such preferred compounds are non-toxic. They do not exhibit single or multiple dose acute or long-term toxicity, mutagenicity (e.g., as determined in a bacterial reverse mutation assay such as an Ames test), teratogenicity, tumorogenicity, or the like, and rarely trigger adverse effects (side effects) when administered at therapeutically effective dosages.

Preferably, administration of such preferred compounds of the invention at certain doses (i.e., doses yielding therapeutically effective in vivo concentrations or preferably doses of 10, 50, 100, 150, or 200 mg/kg administered parenterally or preferably orally) does not result in prolongation of heart QT intervals (i.e., as determined by electrocardiography, e.g., in guinea pigs, minipigs or dogs). When administered daily for 5 or preferably ten days, such doses of such preferred compounds also do not cause liver enlargement resulting in an increase of liver to body weight ratio of more than 100%, preferably not more than 75% and more preferably not more than 50% over matched controls in laboratory rodents (e.g., mice or rats). In another aspect such doses of such preferred compounds also preferably do not cause liver enlargement resulting in an increase of liver to body weight ratio of more than 50%, preferably not more than 25%, and more preferably not more than 10% over matched untreated controls in dogs or other non-rodent mammals.

In yet another aspect such doses of such preferred compounds also preferably do not promote the release of liver enzymes (e.g., ALT, LDH, or AST) from hepatocytes in vivo. Preferably such doses do not elevate serum levels of such enzymes by more than 100%, preferably not by more than 75% and more preferably not by more than 50% over matched untreated controls in laboratory rodents. Similarly, concentrations (in culture media or other such solutions that are contacted and incubated with cells in vitro) equivalent to two, fold, preferably five-fold, and most preferably ten-fold the minimum in vivo therapeutic concentration do not cause release of any of such liver enzymes from hepatocytes into culture medium in vitro above baseline levels seen in media from untreated cells.

Because side effects are often due to undesirable receptor activation or antagonism, preferred compounds of the invention exert their receptor-modulatory effects with high selectivity. This means that they do not bind to certain other receptors (other than CRF receptors) with high affinity, but rather only bind to, activate, or inhibit the activity of such other receptors with affinity constants of greater than 100 nanomolar, preferably greater than 1 micromolar, more preferably greater than 10 micromolar and most preferably greater than 100 micromolar. Such receptors preferably are selected from the group including ion channel receptors, including sodium ion channel receptors, neurotransmitter receptors such as alpha- and beta-adrenergic receptors, muscarinic receptors (particularly m1, m2, and m3 receptors), dopamine receptors, and metabotropic glutamate receptors; and also include histamine receptors and cytokine receptors, e.g., interleukin receptors, particularly IL-8 receptors. The group of other receptors to which preferred compounds do not bind with high affinity also includes GABA_{A} receptors, bioactive peptide receptors (including NPY and VIP receptors), neurokinin receptors, bradykinin receptors (e.g., BK1 receptors and BK2 receptors), and hormone receptors (including thyrotropin releasing hormone receptors and melanocyte-concentrating hormone receptors).

Microsomal In Vitro Half-Life

Compound half-life values (t_{1/2} values) may be determined via the following standard liver microsomal half-life assay. Pooled Human liver microsomes are obtained from XenoTech LLC, 3800 Cambridge St. Kansas's City, Kans., 66103 (catalog #H0610). Such liver microsomes may also be obtained from In Vitro Technologies, 1450 South Rolling Road, Baltimore, Md. 21227, or from Tissue Transformation Technologies, Edison Corporate Center, 175 May Street, Suite 600, Edison, N.J. 08837. Reactions are preformed as follows:

Reagents:

Phosphate buffer: 19 mL 0.1 M NaH₂PO₄, 81 mL 0.1 Na₂HPO₄, adjusted to pH 7.4 with H₃PO₄.

CoFactor Mixture: 16.2 mg NADP, 45.4 mg Glucose-6-phosphate in 4 mL 100 mM MgCl₂. Glucose-6-phosphate dehydrogenase: 214.3 ul glucose-6-phosphate dehydrogenase suspension (Boehringer-Manheim catalog no. 0737224, distributed by Roche Molecular Biochemicals, 9115 Hague Road, P.O. Box 50414, Indianapolis, Ind. 46250) is diluted into 1285.7 ul distilled water.

Starting Reaction Mixture: 3 mL CoFactor Mixture, 1.2 mL Glucose-6-phosphate dehydrogenase.

Reaction:

6 test reactions are prepared, each containing 25 ul microsomes, 5 μL of a 100 uM solution of test compound, and 399 ul 0.1 M phosphate buffer. A seventh reaction is prepared as a positive control containing 25 ul microsomes, 399 ul 0.1 M phosphate buffer, and 5 ul of a 100 uM solution of a compound with known metabolic properties (e.g. DIAZEPAM or CLOZEPINE). Reactions are preincubated at 39° C. for 10 minutes. 71 μL Starting Reaction Mixture is added to 5 of the 6 test reactions and to the positive control, 71 μL 100 mM MgCl₂ is added to the sixth test reaction, which is used as a negative control. At each time point (0, 1, 3, 5, and 10 minutes) 75 μL of each reaction mix is pipetted into a well of a 96-well deep-well plate containing 75 μL ice-cold acetonitrile. Samples are vortexed and centrifuged 10 minutes at 3500 rpm (Sorval T 6000D centrifuge, H1000B rotor). 75 μL of supernatant from each reaction is transferred to a well of a 96-well plate containing 150 μL of a 0.5 uM solution of a compound with a known LCMS profile (internal standard) per well. LCMS analysis of each sample is carried out and the amount of unmetabolized test compound is measured as AUC, compound concentration versus time is plotted, and the tin value of the test compound is extrapolated.

Preferred compounds of the invention exhibit in vitro t_{1/2} values of greater than 10 minutes and less than 4 hours. Most preferred compounds of the invention exhibit in vitro t_{1/2} values of between 30 minutes and 1 hour in human liver microsomes.

MDCK Toxicity Assay

Compounds causing acute cytotoxicity will decrease ATP production by Madin Darby canine kidney (MDCK) cells in the following assay.

MDCK cells, ATCC no. CCL-34 (American Type Culture Collection, Manassas, Va.) are maintained in sterile conditions following the instructions in the ATCC production information sheet. The PACKARD, (Meriden, Conn.) ATP-LITE-M Luminescent ATP detection kit, product no. 6016941, allows measurement ATP production in MDCK cells.

Prior to assay 1 ul of test compound or control sample is pipetted into PACKARD (Meriden, Conn.) clear bottom 96-well plates. Test compounds and control samples are diluted in DMSO to give final concentration in the assay of 10 micromolar, 100 micromolar, or 200 micromolar. Control samples are drug or other compounds having known toxicity properties.

Confluent MDCK cells are trypsinized, harvested, and diluted to a concentration of 0.1×10⁶ cells/ml with warm (37° C.) VITACELL Minimum Essential Medium Eagle (ATCC catalog #30-2003). 100 ul of cells in medium is pipetted into each of all but five wells of each 96-well plate. Warm medium without cells (100 ul) is pipetted in the remaining five wells of each plate to provide standard curve control wells. These wells, to which no cells are added, are used to determine the standard curve. The plates are then incubated at 37° C. under 95% O₂, 5% CO₂ for 2 hours with constant shaking. After incubation, 50 μL of mammalian cell lysis solution is added per well, the wells are covered with PACKARD TOPSEAL stickers, and plates are shaken at approximately 700 rpm on a suitable shaker for 2 minutes.

During the incubation, PACKARD ATP LITE-M reagents are allowed to equilibrate to room temperature. Once equilibrated the lyophilized substrate solution is reconstituted in 5.5 mls of substrate buffer solution (from kit). Lyophilized ATP standard solution is reconstituted in deionized water to give a 10 mM stock. For the five control wells, 10 μL of serially diluted PACKARD standard is added to each of the five standard curve control wells to yield a final concentration in each subsequent well of 200 nM, 100 nM, 50 nM, 25 nM, and 12.5 nM.

PACKARD substrate solution (50 μL) is added to all wells. Wells are covered with PACKARD TOPSEAL stickers, and plates are shaken at approximately 700 rpm on a suitable shaker for 2 minutes. A white PACKARD sticker is attached to the bottom of each plate and samples are dark adapted by wrapping plates in foil and placing in the dark for 10 minutes. Luminescence is then measured at 22° C. using a luminescence counter, e.g. PACKARD TOPCOUNT Microplate Scintillation and Luminescense Counter or TECAN SPECTRAFLUOR PLUS.

Luminescence values at each drug concentration are compared to the values computed from the standard curve for that concentration. Preferred test compounds exhibit luminescence values 80% or more of the standard, or preferably 90% or more of the standard, when a 10 micromolar (μM) concentration of the test compound is used. When a 100 μM concentration of the test compound is used, preferred test compounds exhibit luminescence values 50% or more of the standard, or more preferably 80% or more of the standard.

What is claimed is:

1. A compound of the Formula I 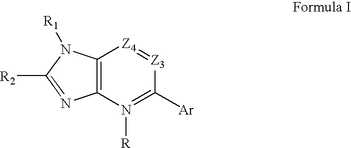
or a pharmaceutically acceptable salt thereof, wherein:
Ar is chosen from:
phenyl which is mono-, di-, or tri-substituted, 1-naphthyl and 2-naphthyl, each of which is optionally mono-, di-, or tri-substituted, and optionally mono-, di-, or tri-substituted heteroaryl, having from 1 to 3 rings, 5 to 7 ring members in each ring and, in at least one ring, from 1 to about 3 heteroatoms selected from N, O, and S;
R is oxygen, methyl, or absent;
R₁ is chosen from optionally substituted alkyl, optionally substituted alkenyl, optionally substituted alkynyl, optionally substituted cycloalkyl, optionally substituted (cycloalkyl)alkyl, optionally substituted mono- or di-alkylamino, optionally substituted alkanoyl, optionally substituted aryl, and optionally substituted heteroaryl, having from 1 to 3 rings, 5 to 7 ring members in each ring and, in at least one ring, from 1 to about 3 heteroatoms selected from N, O, and S;
R₂ is chosen from hydrogen, halogen, hydroxy, amino, cyano, nitro, alkyl, alkoxy, haloalkyl, haloalkoxy, aminoalkyl, and mono- and dialkylamino;
Z₃ is nitrogen or CR₃;
Z₄ is nitrogen or CR₄;
wherein Z₃ and Z₄ are not both nitrogen; and
R₃ and R₄ are independently chosen from hydrogen, halogen, hydroxy, amino, cyano, nitro, optionally substituted alkyl, optionally substituted alkenyl, optionally substituted alkynyl, optionally substituted alkoxy, optionally substituted mono- and di-alkylamino, optionally substituted (cycloalkyl)alkyl, optionally substituted (cycloalkyl)oxy, optionally substituted cycloalkyl, optionally substituted (cycloalkyl)alkoxy, optionally substituted alkylthio, optionally substituted alkylsulfinyl, optionally substituted alkylsulfonyl, optionally substituted mono- and di-alkylcarboxamide, optionally substituted aryl, and optionally substituted heteroaryl, having from 1 to 3 rings, 5 to 7 ring members in each ring and, in at least one ring, from 1 to about 3 heteroatoms selected from the group consisting of N, O, and S.

2. A compound of the Formula I 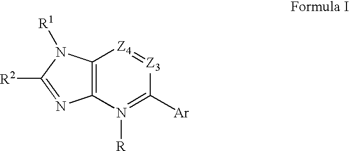
or a pharmaceutically acceptable salt thereof, wherein:
R is oxygen, methyl, or absent;
Ar is chosen from:
phenyl which is mono-, di-, or tri-substituted with R_{A}, and 1-naphthyl, 2-naphthyl, pyridyl, pyrimidinyl, pyrazinyl, pyridizinyl, thienyl, thiazolyl, oxazolyl, isoxazolyl, pyrrolyl, furanyl, and triazolyl, each of which is optionally mono-, di-, or tri-substituted with R_{A};
R₁ is chosen from
i) C₁-C₁₀-carbhydryl, (C₃-C₇cycloalkyl)C₀-C₄-carbhydryl, (C₁C₆)haloalkyl, and mono- and di-(C₁C₆)alkylamino, C₂-C₆alkanoyl; each of which is substituted with 0 or more substituents independently chosen from halogen, hydroxy, amino, oxo, cyano, C₁-C₄alkoxy, and mono- and di(C₁-C₄)alkylamino, and
ii) phenyl which is mono-, di-, or tri-substituted with R_{A}, 1-naphthyl, 2-naphthyl, pyridyl, dihydropyridyl, tetrahydropyridyl, pyrimidinyl, pyrazinyl, pyridizinyl, thienyl, thiazolyl, oxazolyl, isoxazolyl, pyrrolyl, furanyl, and triazolyl, each of which is optionally mono-, di-, or tri-substituted with R_{A};
R₂ is chosen from hydrogen, halogen, hydroxy, amino, cyano, nitro, C₁-C₆alkyl, (C₁-C₆)haloalkyl, (C₁-C₆)haloalkoxy, C₁-C₆alkoxy, amino(C₁-C₆)alkyl, and mono- and di-(C₁-C₆)alkylamino;
Z₃ is nitrogen or CR₃;
Z₄ is nitrogen or CR₄;
wherein Z₃ and Z₄ are not both nitrogen;
and
R₃ and R₄ independently chosen from
i) hydrogen, halogen, hydroxy, cyano, nitro, amino,
ii) C₁-C₆alkyl, C₂-C₆alkenyl, C₂-C₆alkynyl, C₁-C₆alkoxy, mono- and di-C₁-C₆alkylamino, (C₃-C₇cycloalkyl)C₀-C₄alkyl, (C₃-C₇cycloalkyl)C₀-C₄alkoxy, C₁-C₆haloalkyl, C₁-C₆haloalkoxy, —S(O)ₙ(C₁-C₆alkyl), and mono- and di-C₁-C₆alkylcarboxamide, each of which is substituted with 0 or more substituents independently chosen from halogen, amino, hydroxy, oxo, cyano, C₁-C₄alkoxy, and mono- and di-(C₁-C₄)alkylamino; and
iii) phenyl, pyridyl, pyrimidinyl, pyrazinyl, pyridizinyl, thienyl, thiazolyl, oxazolyl, isoxazolyl, pyrrolyl, furanyl, and triazolyl, each of which is substituted with 0 or more substituents independently chosen from halogen, amino, hydroxy, oxo, cyano, C₁-C₄alkoxy, and mono- and di-(C₁-C₄)alkylamino;
R_{A} is independently selected at each occurrence from halogen, cyano, nitro, hydroxy, amino, C₁-C₆haloalkyl, C₁-C₆haloalkoxy, C₁-C₆alkyl substituted with 0-2 R_{B}, C₂-C₆alkenyl substituted with 0-2 R_{B}, C₂-C₆alkynyl substituted with 0-2 R_{B}, C₃-C₇cycloalkyl substituted with 0-2 R_{B}, (C₃-C₇cycloalkyl)C₁-C₄alkyl substituted with 0-2 R_{B}, C₁-C₆alkoxy substituted with 0-2 R_{B}, —NH(C₁-C₆alkyl) substituted with 0-2 R_{B}, —N(C₁-C₆alkyl)(C₁-C₆alkyl) of which each C₁-C₆alkyl is independently substituted with 0-2 R_{B}, —XR_{C}, and Y;
R_{B} is independently selected at each occurrence from halogen, hydroxy, cyano, amino, C₁-C₄alkyl, C₁-C₄alkyl, mono- and di-(C₁-C₄)alkylamino, —S(O)ₙ(alkyl), C₁-C₄haloalkyl, C₁-C₄haloalkoxy, C₁-C₄alkanoyl, mono- and di-C₁-C₄alkylcarboxamide, —XR_{C} and Y;
R_{C} and R_{D}, are independently selected at each occurrence from:
i) hydrogen, and
ii) C₁-C₈alkyl and C₃-C₇cycloalkyl(C₀-C₄alkyl), each of which is substituted with 0 or more substituent(s) independently selected from: oxo, hydroxy, halogen, cyano, amino, C₁-C₆alkoxy, mono- and di-C₁-C₆alkylamino, mono- and di-C₁-C₆alkylcarboxamide, —NHS(O)ₙ(C₁-C₆alkyl), —S(O)ₙ(C₁-C₆alkyl), —S(O)ₙNH(C₁-C₆alkyl), —S(O)ₙN(C₁-C₆alkyl)(C₁-C₆alkyl), and Z;
X is independently selected at each occurrence from —CH₂—, —CHR_{D}—, —O—, —C(═O)—, —C(═O)O—, —S(O)ₙ—, —NH—, —NR_{D}—, —C(═O)NH—, —C(═O)NR_{D}—, —S(O)ₙNH—, —S(O)ₙNR_{D}—, —OC(═S)S—, —NHC(═O)—, —NR_{D}C(═O)—, —NHS(O)ₙ—, and —NR_{D}S(O)ₙ—;
Y and Z are independently selected at each occurrence from: 3- to 7-membered carbocyclic or heterocyclic groups, each of which is substituted with 0 or more substituents independently selected from halogen, oxo, hydroxy, amino, cyano, C₁-C₄alkyl, C₁-C₄alkoxy, mono- and di-C₁-C₆alkylamino, and —S(O)ₙ(alkyl); and
n is independently selected at each occurrence from 0, 1, and 2.

3. A compound or salt according to claim 2 wherein R is absent.

4. A compound or salt according to claim 3, wherein Z₃ is CR₃ and Z₄ is nitrogen.

5. A compound or salt according to claim 3, wherein Z₃ is nitrogen and Z₄ is CR₄.

6. A compound or salt according to claim 3, wherein Z₃ is CR₃ and Z₄ is CR₄.

7. A compound or salt according to any one of claims 4 to 6, wherein
R₁ is chosen from C₁-C₁₀carbhydryl, (C₃-C₇cycloalkyl)C₀-C₄carbhydryl, and (C₁C₆)haloalkyl, each of which is substituted with 0 or more substituents independently chosen from halogen, hydroxy, amino, oxo, cyano, C₁-C₄alkoxy, and mono- and di-(C₁-C₄)alkylamino;
R₂ is chosen from hydrogen, halogen, C₁-C₆alkyl, C₁-C₆alkoxy, C₁-C₄haloalkyl and C₁-C₄haloalkoxy, and mono- and di-C₁-C₄alkylamino; and
R₃ and R₄, when present, are independently chosen from hydrogen, halogen, C₁-C₆alkyl, C₁-C₆alkoxy, C₁-C₄haloalkyl, C₁-C₄haloalkoxy, and mono- and di-C₁-C₆alkylamino.

8. A compound or salt according to claim 7, wherein:
R₁ is chosen from C₁-C₁₀carbhydryl and (C₃-C₇cycloalkyl)C₀-C₄alkyl; each of which is substituted with one or more substituents independently chosen from halogen, hydroxy, amino, oxo, cyano, C₁-C₄alkoxy, and mono- and di-(C₁-C₄)alkylamino;
R₂ is chosen from hydrogen, halogen, C₁-C₆alkyl and C₁-C₄haloalkyl; and
R₃ and R₄, when present, are chosen from hydrogen, halogen, C₁-C₆alkyl, C₁-C₆alkoxy, (C₁-C₄)haloalkyl, (C₁-C₄)haloalkoxy, and mono- and di-C₁-C₄alkylamino.

9. A compound or salt according to any one of claims 4 to 6, wherein:
Ar is phenyl, pyridyl, or pyrimidinyl, each of which is mono-, di-, or tri-substituted with R_{A};
R₁ is chosen from C₁-C₁₀carbhydryl and (C₃-C₇cycloalkyl)C₀-C₄alkyl; each of which is substituted with 0 or more substituents independently chosen from halogen, hydroxy, amino, oxo, cyano, C₁-C₄alkoxy, and mono- and di-(C₁-C₄)alkylamino;
R₂ is chosen from hydrogen, halogen, C₁-C₆alkyl and (C₁-C₄)haloalkyl; and
R₃ and R₄, when present, are chosen from hydrogen, halogen, C₁-C₆alkyl, C₁-C₆alkoxy, (C₁-C₄)haloalkyl, (C₁-C₄)haloalkoxy, and mono- and di-C₁-C₄alkylamino.

10. A compound or salt according to claim 9, wherein:
Ar is chosen from phenyl, pyridyl, and pyrimidinyl, each of which is mono-, di-, or tri-substituted by substituents independently chosen from:
i) halogen, cyano, (C₁-C₄)haloalkyl, (C₁-C₄)haloalkoxy, hydroxy, amino, C₃-C₇cycloalkyl(C₀-C₄alkyl), mono- and di-C₁-C₆alkylamino, —CHO, and —C(═O)CH₃,
ii) C₁-C₆alkoxy and C₁-C₆alkyl which are substituted with 0 or 2 groups independently selected from halogen, hydroxy, cyano, amino, C₁-C₄alkyl, C₁-C₄alkoxy, mono- and di-C₁-C₄alkylamino, (C₁-C₄)haloalkyl, (C₁-C₄)haloalkoxy, and C₂-C₅alkanoyl, and
iii) 3- to 7-membered carbocyclic and heterocyclic groups, each of which is substituted with 0 or more substituents independently selected from halogen, oxo, hydroxy, amino, C₁-C₄alkyl, C₁-C₄alkoxy, and mono- and di-(C₁-C₄)alkylamino;
wherein at least one position ortho to the point of attachment of Ar in Formula I is substituted;
R₁ is chosen from C₁-C₁₀carbhydryl and (C₃-C₇cycloalkyl)C₀-C₄alkyl; each of which is substituted with one or more substituents independently chosen from halogen, hydroxy, amino, oxo, cyano, C₁-C₄alkoxy, and mono- and di-(C₁-C₄)alkylamino;
R₂ is chosen from hydrogen, halogen, C₁-C₆alkyl and (C₁-C₄)haloalkyl; and
R₃ and R₄, when present, are chosen hydrogen, halogen, C₁-C₆alkyl, C₁-C₆alkoxy, (C₁-C₄)haloalkyl, (C₁-C₄)haloalkoxy, mono- and di-C₁-C₄alkylamino.

11. A compound or salt according to claim 10, wherein:
Ar is phenyl, pyridyl, or pyrimidinyl, each of which is mono-, di-, or tri-substituted with substituents independently chosen from:
i) halogen, cyano, (C₁-C₄)haloalkyl, (C₁-C₄)haloalkoxy, hydroxy, amino, C₃-C₇cycloalkylC₀-C₄alkyl, mono- and di-(C₁-C₆)alkylamino, —CHO, and —C(═O)CH₃,
ii) C₁-C₆alkoxy and C₁-C₆alkyl which are substituted with from 0 to 2 groups independently selected from halogen, hydroxy, cyano, amino, C₁-C₄alkyl, C₁-C₄alkoxy, mono- and di-(C₁-C₄)alkylamino, (C₁-C₄)haloalkyl, (C₁-C₄)haloalkoxy, and C₂-C₅alkanoyl, and
iii) phenyl, pyridyl, pyrimidinyl, pyrazinyl, morpholinyl, piperidinyl, piperazinyl, and dioxolanyl, each of which is substituted with 0 or more substituents independently selected from halogen, oxo, hydroxy, amino, C₁-C₄alkyl, C₁-C₄alkoxy, and mono- and di-(C₁-C₄)alkylamino;
wherein at least one position ortho to the point of attachment of Ar in Formula I is substituted; and
R₁ is chosen from C₁-C₁₀alkyl and (C₃-C₇cycloalkyl)C₀-C₄alkyl, each of which is substituted with 0 or more substituents independently chosen from halogen, hydroxy, amino, oxo, cyano, C₁-C₄alkoxy, and mono- and di-(C₁-C₄)alkylamino;
R₂ is chosen from hydrogen, halogen, C₁-C₆alkyl and (C₁-C₄)haloalkyl; and
R₃ and R₄, when present, are chosen hydrogen, halogen, C₁-C₆alkyl, C₁-C₆alkoxy, (C₁-C₄)haloalkyl, and (C₁-C₄)haloalkoxy.

12. A compound or salt according to claim 1 wherein
Ar is phenyl, pyridyl or pyrimidinyl, each of which is mono-, di-, or tri-substituted with substituents independently chosen from halogen, cyano, hydroxy, amino, C₁-C₆alkyl, C₁-C₆alkoxy, (C₁-C₂)haloalkyl, (C₁-C₂)haloalkoxy, C₃-C₇cycloalkylC₀-C₂alkyl, mono- and di-(C₁-C₄)alkylamino, —CHO, and —C(═O)CH₃;
R₁ is chosen from C₁-C₈alkyl and (C₃-C₇cycloalkyl)C₀-C₂alkyl; each of which is substituted with from 0 to 3 substituents independently chosen from halogen, hydroxy, amino, oxo, cyano, C₁-C₄alkoxy, and mono- and di-(C₁-C₄)alkylamino;
R₂ is chosen from hydrogen, halogen, C₁-C₂alkyl and (C₁-C₂)haloalkyl; and
R₃ and R₄, when present, are chosen hydrogen, halogen, C₁-C₆alkyl, C₁-C₆alkoxy, (C₁-C₂)haloalkyl, and (C₁-C₂)haloalkoxy.

13. A compound or salt according to claim 12, wherein
Ar is phenyl, pyridyl or pyrimidinyl, each of which is mono-, di-, or tri-substituted with substituents independently chosen from halogen, cyano, hydroxy, amino, C₁-C₄alkyl, C₁-C₃alkoxy, (C₁-C₂)haloalkyl, (C₁-C₂)haloalkoxy, and mono- and di-(C₁-C₄)alkylamino;
R₁ is chosen from C₁-C₈alkyl which is substituted with from 0 to 3 substituents independently chosen from halogen, hydroxy, amino, oxo, cyano, C₁-C₄alkoxy, and mono- and di-(C₁-C₄)alkylamino;
R₂ is chosen from hydrogen, halogen, C₁-C₂alkyl and (C₁-C₂)haloalkyl; and
R₃ and R₄, when present, are chosen hydrogen, halogen, C₁-C₄alkyl, C₁-C₄alkoxy, (C₁-C₂)haloalkyl, and (C₁-C₂)haloalkoxy.

14. A compound or salt according to claim 13, wherein:
Ar is phenyl, pyridyl or pyrimidinyl, each of which is mono-, di-, or tri-substituted with substituents independently chosen from halogen, amino, C₁-C₄alkyl, C₁-C₃alkoxy, (C₁-C₂)haloalkyl, (C₁-C₂)haloalkoxy, and mono- and di-(C₁-C₂)alkylamino;
R₁ is chosen from C₁-C₈alkyl which is substituted with from 0 to 3 substituents independently chosen from halogen, hydroxy, amino, C₁-C₄alkoxy, and mono- and di-(C₁-C₄)alkylamino;
R₂ is chosen from hydrogen, methyl, and ethyl; and
R₃ and R₄, when present, are chosen hydrogen, halogen, ethyl, methyl, propyl, methoxy, ethoxy, and trifluoromethyl.

15. A compound of Formula V 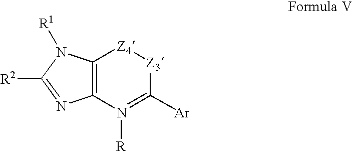
or a pharmaceutically acceptable salt thereof, wherein:
Ar is chosen from phenyl which is mono-, di-, or tri-substituted, 1-naphthyl and 2-naphthyl, each of which is optionally mono-, di-, or tri-substituted, and optionally mono-, di-, or tri-substituted heteroaryl, having from 1 to 3 rings, 5 to 7 ring members in each ring and, in at least one ring, from 1 to about 3 heteroatoms selected from N, O, and S;
R is oxygen, methyl, or absent;
R₁ is chosen from optionally substituted alkyl, optionally substituted alkenyl, optionally substituted alkynyl, optionally substituted cycloalkyl, optionally substituted (cycloalkyl)alkyl, optionally substituted mono- or di-alkylamino, optionally substituted alkanoyl, optionally substituted aryl, and optionally substituted heteroaryl, having from 1 to 3 rings, 5 to 7 ring members in each ring and, in at least one ring, from 1 to about 3 heteroatoms selected from N, O, and S;
R₂ is chosen from hydrogen, halogen, hydroxy, amino, cyano, nitro, alkyl, alkoxy, haloalkyl, haloalkoxy, aminoalkyl, and mono- and dialkylamino;
Z₃′ is CR₃R₃′, NR₃″ or C═O;
Z₄′ is CR₄R₄′, NR₄″ or C═O;
wherein one of Z₃′ or Z₄′ is C═O;
R₃, R₃′, R₄, and R₄′ are independently chosen from hydrogen, halogen, hydroxy, amino, cyano, nitro, optionally substituted alkyl, optionally substituted alkenyl, optionally substituted alkynyl, optionally substituted alkoxy, optionally substituted mono- and di-alkylamino, optionally substituted (cycloalkyl)alkyl, optionally substituted (cycloalkyl)oxy, optionally substituted (cycloalkyl)alkoxy, optionally substituted alkylthio, optionally substituted alkylsulfinyl, optionally substituted alkylsulfonyl, optionally substituted mono- and di-alkylcarboxamide, optionally substituted aryl, and optionally substituted heteroaryl, having from 1 to 3 rings, 5 to 7 ring members in each ring and, in at least one ring, from 1 to about 3 heteroatoms selected from the group consisting of N, O, and S; and
R₃″ and R₄″ are independently chosen from hydrogen, alkyl, aminoalkyl, and haloalkyl.

16. A compound of Formula V: 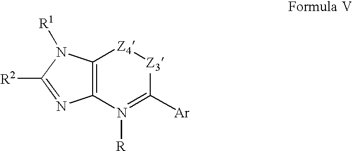
or a pharmaceutically acceptable salt thereof, wherein:
R is oxygen, methyl, or absent;
Ar is chosen from: phenyl, which is mono-, di-, or tri-substituted with R_{A}, and 1-naphthyl, 2-naphthyl, pyridyl, pyrimidinyl, pyrazinyl, pyridizinyl, thienyl, thiazolyl, oxazolyl, isoxazolyl, pyrrolyl, furanyl, and triazolyl, each of which is optionally mono-, di-, or tri-substituted with R_{A};
R₁ is chosen from
i) C₁-C₁₀carbhydryl, (C₃-C₇cycloalkyl)C₀-C₄carbhydryl, (C₁C₆)haloalkyl, and mono- and di-(C₁C₆)alkylamino, C₂-C₆alkanoyl; each of which is substituted with 0 or more substituents independently chosen from halogen, hydroxy, amino, oxo, cyano, C₁-C₄alkoxy, and mono- and di(C₁-C₄)alkylamino, and
ii) phenyl, which is mono-, di-, or tri-substituted with R_{A}, 1-naphthyl, 2-naphthyl, pyridyl, dihydropyridyl, tetrahydropyridyl, pyrimidinyl, pyrazinyl, pyridizinyl, thienyl, thiazolyl, oxazolyl, isoxazolyl, pyrrolyl, furanyl, and triazolyl, each of which is optionally mono-, di-, or tri-substituted with R_{A};
R₂ is chosen from hydrogen, halogen, hydroxy, amino, cyano, nitro, C₁-C₆alkyl, (C₁-C₆)haloalkyl, (C₁-C₆)haloalkoxy, C₁-C₆alkoxy, amino(C₁-C₆)alkyl, and mono- and di-(C₁-C₆)alkylamino;
Z₃′ is CR₃R₃′, NR₃″ or C═O;
Z₄′ is CR₄R₄′, NR₄″ or C═O;
wherein one of Z₃′ or Z₄′ is C═O;
R₃, R₃′, R₄, and R₄′ independently chosen from
i) hydrogen, halogen, hydroxy, cyano, nitro, amino,
ii) C₁-C₆alkyl, C₂-C₆alkenyl, C₂-C₆alkynyl, C₁-C₆alkoxy, mono- and di-C₁-C₆alkylamino, (C₃-C₇cycloalkyl)C₀-C₄alkyl, (C₃-C₇cycloalkyl)C₀-C₄alkoxy, C₁-C₆haloalkyl, C₁-C₆haloalkoxy, —S(O)ₙ(C₁-C₆alkyl), and mono- and di-C₁-C₆alkylcarboxamide, each of which is substituted with 0 or more substituents independently chosen from halogen, amino, hydroxy, oxo, cyano, C₁-C₄alkoxy, and mono- and di-(C₁-C₄)alkylamino; and
iii) phenyl, pyridyl, pyrimidinyl, pyrazinyl, pyridizinyl, thienyl, thiazolyl, oxazolyl, isoxazolyl, pyrrolyl, furanyl, and triazolyl, each of which is substituted with 0 or more substituents independently chosen from halogen, amino, hydroxy, oxo, cyano, C₁-C₄alkoxy, and mono- and di-(C₁-C₄)alkylamino;
R₃″ and R₄″ are independently chosen from hydrogen, C₁-C₆alkyl, amino(C₁-C₆)alkyl, and (C₁-C₆)haloalkyl;
R_{A} is independently selected at each occurrence from halogen, cyano, nitro, hydroxy, amino, C₁-C₆haloalkyl, C₁-C₆haloalkoxy, C₁-C₆alkyl substituted with 0-2 R_{B}, C₂-C₆alkenyl substituted with 0-2 R_{B}, C₂-C₆alkynyl substituted with 0-2 R_{B}, C₃-C₇cycloalkyl substituted with 0-2 R_{B}, (C₃-C₇cycloalkyl)C₁-C₄alkyl substituted with 0-2 R_{B}, C₁-C₆alkoxy substituted with 0-2 R_{B}, —NH(C₁-C₆alkyl) substituted with 0-2 R_{B}, —N(C₁-C₆alkyl)(C₁-C₆alkyl) of which each C₁-C₆alkyl is independently substituted with 0-2 R_{B}, —XR_{C}, and Y;
R_{B} is independently selected at each occurrence from halogen, hydroxy, cyano, amino, C₁-C₄alkyl, mono- and di-(C₁-C₄)alkylamino, —S(O)ₙ(alkyl), C₁-C₄haloalkyl, C₁-C₄haloalkoxy, C₁-C₄alkanoyl, mono- and di-C₁-C₄alkylcarboxamide, —XR_{C}, and Y;
R_{C} and R_{D}, are independently selected at each occurrence from:
i) hydrogen, and
ii) C₁-C₈alkyl and C₃-C₇cycloalkyl(C₀-C₄alkyl), each of which is substituted with 0 or more substituent(s) independently selected from: oxo, hydroxy, halogen, cyano, amino, C₁-C₆alkoxy, mono- and di-C₁-C₆alkylamino, mono- and di-C₁-C₆alkylcarboxamide, —NHS(O)ₙ(C₁-C₆alkyl), —S(O)ₙ(C₁-C₆alkyl), —S(O)ₙNH(C₁-C₆alkyl), —S(O)ₙN(C₁-C₆alkyl)(C₁-C₆alkyl), and Z;
X is independently selected at each occurrence from —CH₂—, —CHR_{D}—, —O—, —C(═O)—, —C(═O)O—, —S(O)ₙ—, —NH—, —NR_{D}—, —C(═O)NH—, —C(═O)NR_{D}—, —S(O)ₙNH—, —S(O)ₙNR_{D}, —OC(═S)S—, —NHC(═O)—, —NR_{D}C(═O)—, —NHS(O)ₙ—, and —NR_{D}S(O)ₙ—;
Y and Z are independently selected at each occurrence from: 3- to 7-membered carbocyclic or heterocyclic groups, each of which is substituted with 0 or more substituents independently selected from halogen, oxo, hydroxy, amino, cyano, C₁-C₄alkyl, C₁-C₄alkoxy, mono- and di-C₁-C₆alkylamino, and —S(O)ₙ(alkyl); and
n is independently selected at each occurrence from 0, 1, and 2.

17. A compound or salt according to claim 16 wherein R is absent.

18. A compound or salt according to claim 17 wherein Z₃′ is NR₃″ and Z₄′ is C═O.

19. A compound or salt according to claim 17 wherein Z₃′ is C═O and Z₄ is NR₄″.

20. A compound or salt according to claim 18 or claim 19, wherein
R₁ is chosen from C₁-C₁₀-carbhydryl, (C₃-C₇cycloalkyl)C₀-C₄-carbhydryl, and (C₁C₆)haloalkyl, each of which is substituted with 0 or more substituents independently chosen from halogen, hydroxy, amino, oxo, cyano, C₁-C₄alkoxy, and mono- and di-(C₁-C₄)alkylamino, and
R₂ is chosen from hydrogen, halogen, C₁-C₆alkyl, C₁-C₆alkoxy, C₁-C₄haloalkyl and C₁-C₄haloalkoxy, and mono- and di-C₁-C₄alkylamino; and
R₃″ and R₄″, when present, are chosen from hydrogen, C₁-C₆alkyl, amino(C₁-C₆)alkyl, and (C₁-C₆)haloalkyl.

21. A compound or salt according to claim 20, wherein:
R₁ is chosen from C₁-C₁₀carbhydryl and (C₃-C₇cycloalkyl)C₀-C₄alkyl; each of which is substituted with one or more substituents independently chosen from halogen, hydroxy, amino, oxo, cyano, C₁-C₄alkoxy, and mono- and di-(C₁-C₄)alkylamino,
R₂ is chosen from hydrogen, halogen, C₁-C₆alkyl and C₁-C₄haloalkyl; and
R₃″ and R₄″, when present, are hydrogen.

22. A compound or salt according to claim 18 or claim 19, wherein:
Ar is phenyl, pyridyl, or pyrimidinyl, each of which is mono-, di-, or tri-substituted with R_{A};
R₁ is chosen from C₁-C₁₀carbhydryl and (C₃-C₇cycloalkyl)C₀-C₄alkyl; each of which is substituted with 0 or more substituents independently chosen from halogen, hydroxy, amino, oxo, cyano, C₁-C₄alkoxy, and mono- and di-(C₁-C₄)alkylamino,
R₂ is chosen from hydrogen, halogen, C₁-C₆alkyl and (C₁-C₄)haloalkyl; and
R₃″ and R₄″ when present are hydrogen.

23. A compound or salt according to claim 22, wherein:
Ar is chosen from phenyl, pyridyl, and pyrimidinyl, each of which is mono-, di-, or tri-substituted by substituents independently chosen from:
i) halogen, cyano, (C₁-C₄)haloalkyl, (C₁-C₄)haloalkoxy, hydroxy, amino, C₃-C₇cycloalkyl(C₀-C₄alkyl), mono- and di-C₁-C₆alkylamino, —CHO, and —C(═O)CH₃,
ii) C₁-C₆alkoxy and C₁-C₆alkyl which are substituted with 0 or 2 groups independently selected from halogen, hydroxy, cyano, amino, C₁-C₄alkyl, C₁-C₄alkoxy, mono- and di-C₁-C₄alkylamino, (C₁-C₄)haloalkyl, (C₁-C₄)haloalkoxy, and C₂-C₅alkanoyl, and
iii) 3- to 7-membered carbocyclic and heterocyclic groups, each of which is substituted with 0 or more substituents independently selected from halogen, oxo, hydroxy, amino, C₁-C₄alkyl, C₁-C₄alkoxy, and mono- and di-(C₁-C₄)alkylamino;
wherein at least one position ortho to the point of attachment of Ar in Formula I is substituted;
R₁ is chosen from C₁-C₁₀carbhydryl and (C₃-C₇cycloalkyl)C₀-C₄alkyl; each of which is substituted with one or more substituents independently chosen from halogen, hydroxy, amino, oxo, cyano, C₁-C₄alkoxy, and mono- and di-(C₁-C₄)alkylamino,
R₂ is chosen from hydrogen, halogen, C₁-C₆alkyl and (C₁-C₄)haloalkyl;
R₃″ and R₄″, when present, are hydrogen.

24. A compound or salt according to claim 23, wherein:
Ar is phenyl, pyridyl, or pyrimidinyl, each of which is mono-, di-, or tri-substituted with substituents independently chosen from:
i) halogen, cyano, (C₁-C₄)haloalkyl, (C₁-C₄)haloalkoxy, hydroxy, amino, C₃-C₇cycloalkylC₀-C₄alkyl, mono- and di-(C₁-C₆)alkylamino, —CHO, and —C(═O)CH₃,
ii) C₁-C₆alkoxy and C₁-C₆alkyl which are substituted with from 0 to 2 groups independently selected from halogen, hydroxy, cyano, amino, C₁-C₄alkyl, C₁-C₄alkoxy, mono- and di-(C₁-C₄)alkylamino, (C₁-C₄)haloalkyl, (C₁-C₄)haloalkoxy, and C₂-C₅alkanoyl, and
iii) phenyl, pyridyl, pyrimidinyl, pyrazinyl, morpholinyl, piperidinyl, piperazinyl, and dioxolanyl, each of which is substituted with 0 or more substituents independently selected from halogen, oxo, hydroxy, amino, C₁-C₄alkyl, C₁-C₄alkoxy, and mono- and di-(C₁-C₄)alkylamino;
wherein at least one position ortho to the point of attachment of Ar in Formula I is substituted; and
R₁ is chosen from C₁-C₁₀alkyl and (C₃-C₇cycloalkyl)C₀-C₄alkyl, each of which is substituted with 0 or more substituents independently chosen from halogen, hydroxy, amino, oxo, cyano, C₁-C₄alkoxy, and mono- and di-(C₁-C₄)alkylamino,
R₂ is chosen from hydrogen, halogen, C₁-C₆alkyl and (C₁-C₄)haloalkyl; and
R₃″ and R₄″, when present, are hydrogen.

25. A compound or salt according to claim 24, wherein
Ar is phenyl, pyridyl or pyrimidinyl, each of which is mono-, di-, or tri-substituted with substituents independently chosen from halogen, cyano, hydroxy, amino, C₁-C₆alkyl, C₁-C₆alkoxy, (C₁-C₂)haloalkyl, (C₁-C₂)haloalkoxy, C₃-C₇cycloalkylC₀-C₂alkyl, mono- and di-(C₁-C₄)alkylamino, —CHO, and —C(═O)CH₃;
R₁ is chosen from C₁-C₈alkyl and (C₃-C₇cycloalkyl)C₀-C₂alkyl; each of which is substituted with from 0 to 3 substituents independently chosen from halogen, hydroxy, amino, oxo, cyano, C₁-C₄alkoxy, and mono- and di-(C₁-C₄)alkylamino,
R₂ is chosen from hydrogen, halogen, C₁-C₂alkyl and (C₁-C₂)haloalkyl; and
R₃″ and R₄″, when present, are hydrogen.

26. A compound or salt according to claim 25, wherein
Ar is phenyl, pyridyl or pyrimidinyl, each of which is mono-, di-, or tri-substituted with substituents independently chosen from halogen, cyano, hydroxy, amino, C₁-C₄alkyl, C₁-C₃alkoxy, (C₁-C₂)haloalkyl, (C₁-C₂)haloalkoxy, and mono- and di-(C₁-C₄)alkylamino;
R₁ is chosen from C₁-C₈alkyl which is substituted with from 0 to 3 substituents independently chosen from halogen, hydroxy, amino, oxo, cyano, C₁-C₄alkoxy, and mono- and di-(C₁-C₄)alkylamino, and
R₂ is chosen from hydrogen, halogen, C₁-C₂alkyl and (C₁-C₂)haloalkyl.

27. A compound or salt according to claim 26, wherein:
Ar is phenyl, pyridyl or pyrimidinyl, each of which is mono-, di-, or tri-substituted with substituents independently chosen from halogen, amino, C₁-C₄alkyl, C₁-C₃alkoxy, (C₁-C₂)haloalkyl, (C₁-C₂)haloalkoxy, and mono- and di-(C₁-C₂)alkylamino;
R₁ is chosen from C₁-C₈alkyl which is substituted with from 0 to 3 substituents independently chosen from halogen, hydroxy, amino, C₁-C₄alkoxy, and mono- and di-(C₁-C₄)alkylamino; and
R₂ is chosen from hydrogen, methyl, and ethyl.

28. A compound according to claim 1 which is 1-(1-ethyl-propyl)-5-(2-methoxy-4-trifluoromethoxy-phenyl)-6-methyl-1H-imidazo[4,5-b]pyrazine, or a pharmaceutically acceptable salt thereof.

29. A compound according to claim 1 which is 1-(1-ethyl-propyl)-5-(2-methoxy-4-trifluoromethoxy-phenyl)-2,6-dimethyl-1H-imidazo[4,5-b]pyrazine, or a pharmaceutically acceptable salt thereof.

30. A compound according to claim 1 which is selected from:
1-(1-ethyl-propyl)-5-(2-methoxy-4-trifluoromethoxy-phenyl)-6-methyl-1H-imidazo[4,5-b]pyrazine;
1-(1-ethyl-propyl)-5-(2,6-dimethoxy-4-chloro-phenyl)-6-methyl-1H-imidazo[4,5-b]pyrazine;
5-(2,6-dimethoxy-pyridin-3-yl)-1-(1-ethyl-propyl)-6-methyl-1H-imidazo[4,5-b]pyrazine;
1-(1-ethyl-propyl)-5-(2-methoxy-6-trifluoromethyl-pyridin-3-yl)-6-methyl-1H-imidazo[4,5-b]pyrazine;
1-(1-ethyl-propyl)-5-(2-methoxy-6-isopropyl-pyridin-3-yl)-6-methyl-1H-imidazo[4,5-b]pyrazine;
1-(1-ethyl-propyl)-5-(2-chloro-4-methoxy-phenyl)-6-methyl-1H-imidazo[4,5-b]pyrazine;
1-(1-ethyl-propyl)-5-(2-methoxy-4-trifluoromethoxy-phenyl)-6-methyl-1H-imidazo[4,5-b]pyridine;
{5-[1-(1-ethyl-propyl)-6-methyl-1H-imidazo[4,5-b]pyrazin-5-yl]-4-methoxy-pyridin-2-yl}-dimethyl-amine;
1-(1-ethyl-propyl)-5-(2-methoxy-4-trifluoromethoxy-phenyl)-6-ethyl-1H-imidazo[4,5-b]pyrazine;
5-(2,4-dimethoxy-pyrimidin-5-yl)-1-(1-ethyl-propyl)-6-methyl-1H-imidazo[4,5-b]pyrazine;
1-(1-ethyl-propyl)-5-(2-trifluoromethyl-4-chloro-phenyl)-6-methyl-1H-imidazo[4,5-b]pyrazine;
1-(1-ethyl-propyl)-5-(2-chloro-4-trifluoromethyl-phenyl)-6-methyl-1H-imidazo[4,5-b]pyrazine;
1-(1-ethyl-propyl)-5-(2,4-dichloro-phenyl)-6-methyl-1H-imidazo[4,5-b]pyrazine;
1-(1-ethyl-propyl)-5-(2,4-dichloro-phenyl)-6-ethyl-1H-imidazo[4,5-b]pyrazine;
1-(1-ethyl-propyl)-5-(2-methoxy-4-trifluoromethoxy-phenyl)-6-chloro-1H-imidazo[4,5-b]pyrazine;
1-(1-propyl-butyl)-5-(2-methoxy-4-trifluoromethoxy-phenyl)-6-methyl-1H-imidazo[4,5-b]pyrazine;
1-(1-propyl-butyl)-5-(2-methoxy-6-isopropyl-pyridin-3-yl)-6-methyl-1H-imidazo[4,5-b]pyrazine;
1-(1-propyl-butyl)-5-(2-chloro-4-trifluoromethyl-phenyl)-6-methyl-1H-imidazo[4,5-b]pyridine; and
1-(1-propyl-butyl)-5-(2,4-dichloro-phenyl)-6-methyl-1H-imidazo[4,5-b]pyridine; and
the pharmaceutically acceptable salts thereof.

31. A compound or salt according to claim 1 or claim 15 wherein, in a standard in vitro CRF receptor binding assay the compound exhibits an IC₅₀ value for CRF receptors of less than or equal to 1 micromolar.

32. A compound or salt according to claim 31 wherein, in a standard in vitro CRF receptor binding assay the compound exhibits an IC₅₀ value for CRF receptors of less than or equal to 100 nanomolar.

33. A compound or salt according to claim 32 wherein, in a standard in vitro CRF receptor binding assay, the compound exhibits an IC₅₀ value for CRF receptors of less than or equal to 10 nanomolar.

34. A method for treating anxiety, depression, or stress comprising administering to a patient in need of such treatment a therapeutically effective amount of a compound or salt according to claim 1 or claim 15.

35. A method for treating irritable bowel syndrome or Crohn's disease, comprising administering to a patient in need of such treatment a therapeutically effective amount of a compound or salt according to claim 1 or claim 15.

36. A pharmaceutical composition comprising a pharmaceutically acceptable carrier and a compound or salt of claim 1 or claim 15.

37. A pharmaceutical composition according to claim 36, wherein the composition is formulated as an injectable fluid, an aerosol, a cream, a gel, a tablet, a pill, a capsule, a syrup or a transdermal patch.

38. A package comprising a pharmaceutical composition of claim 37 in a container and further comprising indicia comprising at least one of:
instructions for using the composition to treat a patient suffering from anxiety, or
instructions for using the composition to treat a patient suffering from stress, or
instructions for using the composition to treat a patient suffering from depression.

39. A package comprising a pharmaceutical composition of claim 37 in a container and further comprising at least one of: instructions for using the composition to treat a patient suffering from irritable bowel syndrome or instructions for using the composition to treat a patient suffering from Crohn's disease.

40. A method for demonstrating the presence or absence of CRF 1 receptors in a biological sample, said method comprising:
a) contacting the biological sample with a labeled compound according to claim 1 or claim under conditions that permit binding of the labeled compound to a CRF1 receptor;
b) separating unbound labeled compound from bound labeled compound; and
c) detecting the labeled compound in the biological sample, and therefrom determining the presence or absence of CRF1 receptors in the sample.

41. The method of claim 40 wherein the labeled compound is detected using autoradiography.

42. A method of inhibiting the binding of CRF to a CRF1 Receptor, which method comprises:
contacting a solution comprising CRF and a compound or salt of claim 3 with a cell expressing the CRF1 receptor, wherein the compound or salt is present in the solution at a concentration sufficient to inhibit in vitro CRF binding to IMR32 cells.

43. The method of claim 42 wherein the cell expressing the CRF receptor is a neuronal cell that is contacted in vivo in an animal, and wherein the solution is a body fluid of said animal.

44. The method of claim 43 wherein the animal is a human patient.
